(19) United States
(12) Patent Application Publication
(10) Pub. No.: US 2009/0215783 A1
(43) Pub. Date: Aug. 27, 2009

(54) COMPOSITION FOR INDUCTION OR INHIBITION OF STEM CELL DIFFERENTIATION

(75) Inventor: Se Woong Oh, Suwon (KR)

Correspondence Address: SEED INTELLECTUAL PROPERTY LAW GROUP PLLC, 701 FIFTH AVE, SUITE 5400, SEATTLE, WA, 98104, US

(73) Assignee: CHOONGWAE PHARMA CORPORATION, Seoul (KR)

(21) Appl. No.: 12/302,766

(22) PCT Filed: May 30, 2007
(86) PCT No.: PCT/KR2007/002621

§ 371 Date: Nov. 26, 2008

Related U.S. Application Data
(60) Provisional application No. 60/803,494, filed on May 30, 2006.

(51) Int. Cl. A61K 31/53; A61P 35/00; C07D 487/04; C07D 401/14; C07D 471/04; A61K 31/4985

(52) U.S. Cl. 514/243; 435/377; 544/184; 544/350; 514/249

(57) ABSTRACT

The present invention relates to composition and methods for inducing or inhibiting differentiation of stem cells. The invention also relates to applications in the treatment of medical conditions, e.g., osteoporosis, bone fracture, bone injuries, myocardiac infarction, cardiomyopathy, degenerative muscle diseases, myopathy, and urinary incontinence.

### TECHNICAL FIELD

The present invention relates to composition and methods for inducing or inhibiting differentiation of stem cells. The invention also relates to applications in the treatment of medical conditions, e.g., osteoporosis, bone fracture, bone injuries, myocardiac infarction, cardiomyopathy, degenerative muscle diseases, myopathy, and urinary incontinence.

### BACKGROUND ART

Stem cells are cells that have the ability to self replicate for indefinite periods and have the potential to develop into mature cells that have specialized functions such as heart cells, nerve cells, bone cells, muscle cells, blood cells and pancreatic beta cells. There are several types or sources of stem cells. Embryonic stem (ES) cells are stem cells derived from the inner cell mass of a blastocyst and are pluripotent; i.e., they can differentiate into cells derived from all three primary germ layers: ectoderm, endoderm or stem cell mesoderm (Keller, Genes Develop, 2005, 19, 1129-55). Adult stem cells are undifferentiated cells that reproduce daily to provide certain specialized cells. Adult stem cells have been identified throughout the body including in bone marrow, peripheral blood, brain, spinal cord, liver and pancreas and they have more limited potential than ES cells. Typically, adult stem cells are multipotent cells committed to differentiate into cells that contribute to the function of the tissue from which they originated. However, adult stem cells have been identified with the potential to differentiate into specialized cells of unrelated tissues, including cells derived from a different embryonic germ layer, under certain conditions (Bhatia R and Hare J M, Congest Heart Fail. 2005, 11, 87-91; Weissberg P L and Qasim A, Heart, 2005, 91, 696-702).

The Wnt family of genes encodes for over twenty cysteine-rich secreted glycoproteins that act by binding to Frizzled (Fzd) receptors on target cells. Binding of Wnt to Fzd activates Disheveled (Dvl), leading to the inactivation of Glycogen synthase kinase-3beta (GSK-3beta), a cytoplasmic serine-threonine kinase. The GSK-3beta target beta-catenin is stabilized, translocates to the nucleus, and activates TCF (T cell factor)-dependent transcription on specific promoters (reviewed by Dierick and Bejsovec, 1999; Wodarz and Nusse, 1998). Wnt signaling directs cell fate determination in various tissues, including kidney (Labus et al., Wound Repair Regen, 1998, 6, 58-64; Vainio and Uusitalo, Pediatr Nephrol, 2000, 15, 151-6), CNS (Patapoutian and Reichardt, Curr Opin Neurobiol, 2000, 10, 392-9), hematopoietic (Van Den Berg et al., Blood, 1998, 92, 3189-202), and skeletal muscle (Cossu and Borello, EMBO J, 1999, 18, 6867-72). Moreover, Wnt signaling is implicated in postnatal wound healing and tissue regeneration in zebrafish and hydra (Hobmayer et al., Nature, 2000, 407, 186-9; Labus et al., Wound Repari Regen, 1998, 6, 58-64; Poss et al., Dev Dyn, 2000, 219, 282-6). Wnt signaling has been suggested to be involved in regulation of bone mass and bone formation. A loss of function mutation in LRP5 was found to associate with osteoporosis-pseudoglioma syndrome, an autosomal recessive disorder (Gong Y et al. Cell, 2001, 107, 513-23, Kato M et al. J Cell Biol, 2002, 157, 303-14). Moreover, a Gly171-to-Val substitution mutation in LRP5 results in a high bone mass phenotype (Boyden L M et al. N Engl J Med, 2002, 346, 1513-21). These phenotypes associated with the loss of function or substitution mutations of LRP5 indicate that Wnt signaling might be involved in modulating the regulation of bone mass and bone formation (Westendorf J J et al. Gene, 2004, 341, 19-39). During osteogenesis, pluripotent mesenchymal stem cells differentiate into preosteoblasts, which then differentiate into mature osteoblasts that deposit the necessary components to form bone matrix and subsequent mineralization. Upon differentiation into osteoblasts, the cells express differentiation-related phenotypes such as a high level of alkaline phosphatase (ALP), parathyroid hormone receptor, type I collagen, osteocalcin, matrix extracellular phosphoglycoprotein (MEPE), and bone sialoproteins. In cultured cells, Bain et al. (Biochem Biophys Res Commun, 203, 301, 84-91) described that stimulation of canonical Wnt signaling using constitutively active forms of beta-catenin induces the activity of ALP. Human mesenchymal stem cells (hMSCs) are pluripotent cells from the bone marrow, which can be expanded in vitro and differentiated into the osteogenic, chondrogenic, and adipogenic lineages (Pittenger M F et al, Science, 1999, 284, 143-7). MSCs were initially identified as the fibroblastic adherent fraction of bone marrow aspirates (Castro-Malaspina H. et al, Blood, 1980, 56, 289-301) and are also called colony forming units-fibroblasts (CFU-F), marrow stromal cells, bone marrow mesenchymal cells, or mesenchymal progenitor cells. In vitro osteogenic differentiation of hMSCs recapitulates many of the developmental steps during normal in vivo osteogenesis. For instance, in the presence of dexamethasone and beta-glycerol phosphate, hMSCs express osteogenic markers such as bone-specific alkaline phosphatase (ALP) and they deposit an extracellular matrix, which becomes mineralized under appropriate culture conditions (Caplan A I and Bruder S P., Trends Mol Med, 2001, 7, 259-64) Because of their ready availability and well-established in vitro culturing protocols, hMSCs have been the source of cells in autologous bone and cartilage tissue engineering (Bianco P and Robey P G., Nature, 2001, 414, 118-21).

Wnt proteins initiate myogenesis in explants of mouse paraxial mesoderm by activating expression of Myf5 and MyoD (Tajbakhsh et al., Development, 1998, 121, 4077-83). Myogenesis in presomitic mesoderm and early somites is inhibited by the Wnt antagonist soluble Frizzled-related protein 3 (sFRP3/Frzb1) (Borello et al., Development, 1999, 126, 4247-55). Therefore, Wnt signaling appears to be necessary and, in some instances, sufficient to induce and maintain the myogenic program in embryonic precursor cells.

The Wnt/β-catenin pathway normally regulates expression of a range of genes involved in promoting proliferation and differentiation. Many of these genes, including cyclin D1 (Shtutman et al., “The cyclin D1 gene is a target of the beta-catenin/LEF-1 pathway,” Proc. Natl. Acad. Sci. USA 96:5522-27 (1999); Tetsu et al., “beta-catenin regulates expression of cyclin D1 in colon carcinoma cells,” Nature 398:422-26 (1999)) and c-myc (He et al., “Identification of c-MYC as a target of the APC pathway,” Science 281:1509-12 (1998)) which play critical roles in cell growth, proliferation, and differentiation. The present invention provides agents which regulates the differentiation of adult and embryonic stem cells, and provides further related advantages as described in detail below.

### DISCLOSURE OF INVENTION

### Technical Problem

In brief, the present invention provides agents that induce or inhibit the differentiation of stem cells and methods for their use.

### Technical Solution

In one aspect, the invention provides methods whereby inducing and promoting osteogenesis of bane marrow stem cells, while in a related aspect the invention provides compounds useful in the method. The invention is directed to methods and compositions that address several problems related to bone remodeling, such as osteoporosis and other bone diseases. The invention also provides for the use of compositions to aid in the healing of fractures or other injuries or abnormalities of bone. The invention provides a process for stimulating or enhancing osteoblast mineralization in a mammalian subject comprising administering to the subject an effective amount of the composition.

In another aspect, the invention provides compositions and methods for inhibiting the differentiation of osteogenesis of bone marrow stem cells.

In another aspect, the invention provides compositions and methods for inducing and directing the differentiation of stem cells into cells of a myocardiac lineage. The present invention provides methods of inducing cardiomyogenesis. Mammalian cells are contacted with a compound whereupon the mammalian cell differentiates into a cell of a myocardiac lineage. The contacting can be conducted in vivo or in vitro. In view of their ability to induce cardiomyogenesis, the compounds are useful for treating cardiac muscle disorders, such as cardiomyopathy and arrhythmia, and for repairing heart muscle tissue damage such as myocardiac infarction resulting from a heart attack, for example.

In another aspect, the invention provides compositions and methods for inhibiting the differentiation of stem cells into myocardiac lineage.

In another aspect, the invention provides compositions and methods for inducing and directing the differentiation of stem cells into cells of a skeletal or smooth muscle cells. Mammalian cells are contacted with a compound whereupon the mammalian cell differentiates into a cell of a myocytic lineage. The step of contacting can be in vivo or in vitro. In view of their ability to induce myogenesis, the compounds are useful for treating degenerative muscle disease, such as muscular dystrophy or myopathy or urinary incontinence.

In another aspect, the invention provides compositions and methods for inhibiting the differentiation of myogenesis of stem cells.

The method of the invention may be used to treat various medical conditions. For instance, in various aspects of the invention: the composition is within a cell, and the agent increases the likelihood that the cell will differentiate; the composition is within a cell, and the agent increases the likelihood that the cell will proliferate.

The composition may be in vivo or ex vivo. In one aspect, the composition is ex vivo and the composition further comprises a stem cell. In another aspect the composition is in vivo and the composition is within a mammal, e.g., a mouse.

In another aspect, the present invention provides a method for modulating cell proliferation, comprising: (a) providing a cell population under conditions where a proportion of the population will proliferate and a proportion of the population will differentiate; and (b) adding a chemical agent to the population, where the agent causes an increase in the proportion of the cells that proliferate relative to the proportion of the cells that differentiate. In various optional embodiments of the method further includes adding an agent to the population that activates a Wnt pathway; the cell population is a population of stem cells; the method is performed ex vivo; the method further includes adding an agent that causes differentiation of the cell population where, e.g., the cells in the population differentiate to form osteoblasts, osteocytes, cardiomyocytes, skeletal muscle cells, blood cells, or the cells in the population differentiate to form neuron cells.

In another aspect, the present invention provides a method for maintaining a stem cell in an undifferentiated state, comprising contacting the stem cell with an agent that inhibits cell differentiation or promotes cell proliferation in an amount effective to maintain the stem cell in an undifferentiated state.

In the methods and compositions of the present invention, the chemical agent can be selected from compounds of formula (I):

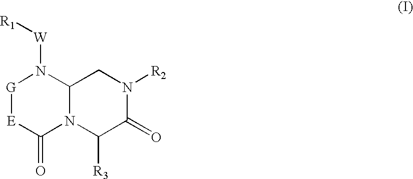

wherein:

E is -(ZR₄)— or —(C═O)—;

G is nothing, —(XR₅)—, or —(C═O)—;

W is —Y(C═O)—, —(C═O)NH—, —(SO₂)— or nothing;

Y is oxygen or sulfur;

X or Z is independently nitrogen or CH;

R₁, R₂, R₃, R₄, and R₅ are the same or different and independently selected from the group consisting of:

an amino acid side chain moiety;

C₁₋₁₂alkyl or substituted C₁₋₁₂alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;

C₁₋₆alkoxy;

C₆₋₁₂aryl or substituted C₆₋₁₂aryl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl;

monocyclic aryl-alkyl having 5 to 7 ring members, which may have 1 to 2 heteroatoms selected from nitrogen, oxygen or sulfur, or substituted monocyclic aryl-alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

bicyclic aryl-alkyl having 8 to 10 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;

tricyclic aryl-alkyl having 5 to 14 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;

arylC₁₋₄alkyl or substituted arylC₁₋₄alkyl having one or more substituents independently selected from amino, amidino, guanidino, hydrazino, C₁₋₄alkylamino, C₁₋₄ dialkylamino, C₃₋₆cycloalkyl, halogen, perfluoroC₁₋₄alkyl, C₁₋₆alkyl, C₁₋₃alkoxy, nitro, carboxyl, cyano, sulfuryl, hydroxyl, amide, C₁₋₆alkyloxyC₁₋₆acyl and morphorlinylC₁₋₆ alkyl;

cycloalkylalkyl or substituted cycloalkylalkyl having one or more substituents independently selected from amino, amidino, guanidino, hydrazino, C₁₋₄alkylamino, C₁₋₄ dialkylamino, halogen, perfluoro C₁₋₄alkyl, C₁₋₄alkyl, C₁₋₃alkoxy, nitro, carboxyl, cyano, sulfuryl and hydroxyl; and

cycloalkyl or substituted cycloalkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl.

In certain embodiments, R₁, R₂, R₃, R₄, and R₅ are the same or different and independently selected from the group consisting of:

C₁₋₁₂alkyl or substituted C₁₋₁₂alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;

C₁₋₆alkoxy;

cycloalkylC₁₋₃alkyl;

cycloalkyl;

phenyl or substituted phenyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;

phenylC₂₋₄alkyl or phenylC₂₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, sulfide, hydroxyl;

naphthyl or substituted naphthyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;

naphthylC₁₋₄alkyl or naphthylC₁₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₄alkyl, C₁₋₃alkoxy, nitro, carboxyl, cyano, sulfuryl, hydroxyl;

benzyl or substituted benzyl having one or more substituents independently selected from amino, amidino, guanidino, hydrazino, C₁₋₄alkylamino, C₁₋₄dialkylamino, halogen, perfluoro C₁₋₄alkyl, trifluoroC₁₋₄alkyl, C₁₋₆alkyl, C₁₋₃alkoxy, nitro, carboxyl, cyano, sulfuryl and hydroxyl;

bisphenylmethyl or substituted bisphenylmethyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

benzylphenyl amide, or substituted benzylphenyl amide having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

pyridyl or substituted pyridyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

pyridylC₁₋₄alkyl, or substituted pyridylC₁₋₄alkyl having one or more substituents independently selected from amino, amidino, guanidino, hydrazino, C₁₋₄alkylamino, C₁₋₄ dialkylamino, halogen, perfluoro C₁₋₄alkyl, C₁₋₄alkyl, C₁₋₃alkoxy, nitro, carboxyl, cyano, sulfuryl and hydroxyl;

pyrimidylC₁₋₄alkyl, or substituted pyrimidylC₁₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

triazin-2-ylC₁₋₄alkyl, or substituted triazin-2-ylC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

imidazolylC₁₋₄alkyl or substituted imidazolylC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

benzothiazolinC₁₋₄alkyl or substituted benzothiazolinC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

phenoxazinC₁₋₄alkyl;

benzyl p-tolyl ether;

phenoxybenzyl;

N-amidinopiperazinyl-N—C₁₋₄alkyl;

quinolineC₁₋₄alkyl;

N-amidinopiperazinyl;

N-amidinopiperidinylC₁₋₄alkyl;

4-aminocyclohexylC₁₋₂alkyl; and

4-aminocyclohexyl.

In certain embodiments, E is -(ZR₄)- and G is —(XR₅)—, wherein Z is CH and X is nitrogen, and the compound has the following general formula (II):

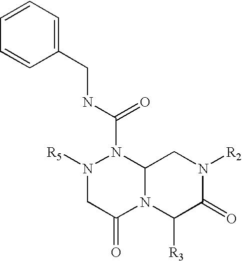

wherein R₂, R₃, and R₅ are as defined as in formula (I).

In certain embodiments, the compound has the following general formula (III):

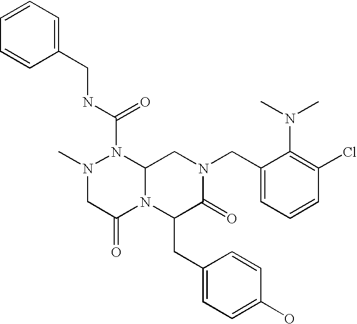

In certain embodiments, E is -(ZR₄)- and G is nothing, wherein Z is nitrogen, and the compound has the following general formula (IV):

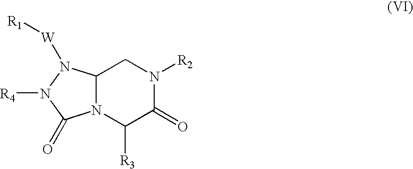

wherein R₁, R₂, R₃, R₄, and W are as defined in formula (I).

In certain embodiments, E is -(ZR₄)- and G is —(XR₅)—, wherein Z and X are independently CH, and the compound has a structure of Formula (V):

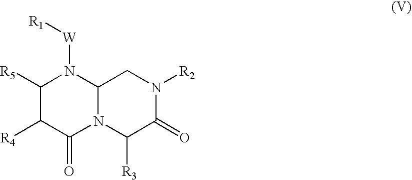

wherein R₁, R₂, R₃, R₄, R₅, and W are as defined in formula (a).

In certain embodiments, the compound has the following general formula (VI):

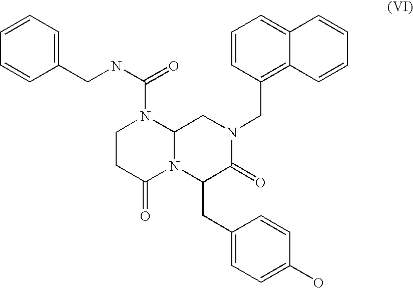

These and related aspects of the present invention are described in further detail below.

### BRIEF DESCRIPTION OF THE DRAWINGS

FIG. 1. A general synthetic scheme for preparing reverse-turn mimetics of the present invention.

FIG. 2. Compound A (Serial No. 111 in Table 2) inhibits β-catenin/TCF transcription. Compound A selectively inhibits a β-catenin/TCF reporter gene construct with an IC₅₀ of 1.455 μM. SW480 cells (10⁵) were transfected with β-catenin/TCF luciferase construct. Cells were treated with Compound A (1-50 μM). 24 hours after the treatment lysates were prepared and subjected to dual luciferase assay.

FIG. 3. Compound B (Serial No. 10 in Table 2) inhibits β-catenin/TCF transcription. Compound B selectively inhibits a β-catenin/TCF reporter gene construct with an IC₅₀ of 6.978 μM. SW480 cells (105) were transfected with β-catenin/TCF luciferase construct. Cells were treated with Compound B (1-50 μM). 24 hours after treatment lysates were prepared and subjected to dual luciferase assay.

FIGS. 4˜7. Modulation of osteogenesis of stem cells by COMPOUNDS.

FIG. 4. Compound B inhibits differentiation of human bone marrow-derived mesenchymal stem cell (hBMMSC) into osteocytic lineage. Compound B was added into the hBMMSC cell culture and cellular alkaline phosphatase (ALP) activity was measured. Compound B at 20 μM strongly inhibited cellular alkaline phosphatase activity. Compound C (Serial No. 82 in Table 2) and D (Serial No. 66 in Table 2) significantly enhanced ALP level (200% and 202% respectively) when it was treated to hBMMSC at 20 μM.

FIG. 5. Compound C and D also strongly induced mineralization of hBMMSC when it was co-treated with osteogenic induction cocktails (OIC).

FIG. 6. When various concentrations of Compound C and D were treated, both compounds showed osteogenesis inducing activity (stimulation of mineralization) even at the 0.5 μM.

FIG. 7. ALP mRNA expression was increased by treatment with Compound C and D in hBMMSC.

FIGS. 8˜14. Modulation of cardiomyogenesis of stem cells by COMPOUNDS.

FIG. 8. A vector construct containing 1.8 kb promoter sequence of the mouse alpha-MHC gene between EcoRI and SalI sites of pEGFP-1.

FIG. 9. Compound E (Serial No. 71 in Table 2) treatment (10 μM) enhanced expression of fluorescence after treatment when it was observed under the fluorescence microscope. And its level was significantly enhanced at Day 5 compared with DMSO control.

FIG. 10. FACS analysis of DMSO treated embryoid bodies (EB). Cell population expressing fluorescence (alpha-MHC positive) was 0.54%.

FIG. 11. FACS analysis of Compound E (10 μM) treated embryoid bodies (EB). Cell population expressing fluorescence (alpha-MHC positive) was 2.50%, which amounts to 4.6 fold increase compared to DMSO treated EB.

FIG. 12. FIG. 12 shows the fold increase of expression level of EGFP (Enhanced Green Fluorescence Protein) in Compound E-treated group compared with DMSO-treated control group. Cardiomyogenesis differentiation promoting activity of Compound E was concentration-dependent and its effect was distinctive even at the 0.3 μM.

FIG. 13. Compound E strongly increased the expression level of ANP and Nkx2.5 mRNA in the ES cells when the ES cells were treated for 7 and 10 days compared with DMSO control.

FIG. 14. Compound B treatment reduced expression of fluorescence (0.26: 1=Compound B: DMSO control) when the mouse embryonic stem cells were exposed to Compound B at 10 μM for 5 days.

FIG. 15. Modulation of myogenesis by Compound F (Serial No. 112 in Table 2).

A. C2C12 myoblasts undergo differentiation to form extensive myotubes when these cells were incubated for 3 days in differentiation medium (DM) containing 2% horse serum.

B. C2C12 cells grown in growth medium (GM) which were treated with Compound F shows extensive myotube formation.

C. C2C12 cells grown in growth medium (GM) without compound F did not show myotube formation.

D. Compound F treatment also significantly increased expression of MyoD and Myf5 proteins.

FIG. 16. Modulation of myogenesis by COMPOUNDS with or without wnt or CBP or p300.

A. Wnt1 conditioned medium was treated with or without test Compound B and F. Myf-5 expression was increased by the treatment of Wnt1 in C2C12 cells and its expression level was further enhanced by co-treatment of Compound F. Compound B treatment reduced expression of Myf-5 in C2C12 cells and it also abolished Myf-5 enhancing activity of Wnt1 when it was co-treated with Wnt1.

B. CBP or p300 with or without test compounds were exposed to the C2C12 cells and cellular expression level of Myf5 protein was determined. Compound F at 5 and 10 μM dose-dependently increased expression of Myf5 compared with DMSO control. Compound B at 5 μM and 10 μM dose-dependently decreased expression of Myf5 compared with DMSO control. And co-treatment of p300 with Compound B recovers decrease of Myf5 expression by Compound B but it was not recovered by co-treatment of CBP.

### BEST MODE FOR CARRYING OUT THE INVENTION

The present invention provides agents that inducing or inhibiting differentiation of stem cells, and methods related thereto.

More specific details of these methods and agents are provided below. However, before providing these details, the following definitions are provided to assist the reader in understanding the present disclosure.

### Definitions

A “Stem cell,” as used herein, refers to any self-renewing pluripotent cell or multipotent cell or progenitor cell or precursor cell that is capable of differentiating into multiple cell types. Stem cells suitable for use in the methods of the present invention include those that are capable of differentiating into cells of osteogenic lineage e.g., osteoblast, osteocytes or myocardiac lineage e.g., cardiomyocytes or skeletal muscle cells, smooth muscle cells, blood cells, or neurons etc.

The term “differentiation,” as used herein, refers to a developmental process whereby cells become specialized for a particular function, for example, where cells acquire one or more morphological characteristics and/or functions different from that of the initial cell type. The term “differentiation” includes both lineage commitment and terminal differentiation processes. Differentiation may assessed, for example, by monitoring the presence or absence of lineage markers, using FACS analysis, immunohistochemistry or other procedures known to a worker skilled in the art. Differentiated progeny cells derived from progenitor cells may be, but not necessarily, related to the same germ layer of tissue as the source tissue of the stem cells. For example, neural progenitor cells and muscle progenitor cells can differentiate into homatopoietic cell lineages.

Osteogenesis, as used herein, refers to proliferation of bone cells and growth of bone tissue (i.e., synthesis and deposit of new bone matrix). Osteogenesis also refers to differentiation or transdifferentiation of progenitor or precursor cells into bone cells (i.e., osteoblasts). Progenitor or precursor cells can be pluripotent stem cells such as mesenchymal stem cells. Progenitor or precursor cells can be cells pre-committed to an osteoblast lineage (e.g., pre-osteoblast cells) or cells that are not pre-committed to an osteoblast lineage (e.g., pre-adipocytes or myoblasts).

The term “cardiomyogenesis,” as used herein, refers to the differentiation of progenitor or precursor cells into cardiac muscle cells (i.e., cardiomyocytes) and the growth of cardiac muscle tissue. Progenitor or precursor cells can be pluripotent stem cells such as embryonic stem cells. Progenitor or precursor cells can be cells pre-committed to a myocardiac lineage (e.g., precardiomyocyte cells) or cells that are not pre-committed (e.g., multipotent adult stem cells).

The term “Cancer stem cells” represents a subpopulation of cells within a tumor which is capable of initiating new tumors following a prolonged period of remission. Presumably this occurs because cancer stem cells have unique properties such as longevity, quiescence and self-renewal, similar to normal tissue stem cells. Self-renewal is the process by which a stem cell produces a similar daughter cell by symmetric division.

β-catenin refers to a protein that is well known in the art, see, e.g., Morin, P. J., Bioessays 21:1021-30 (1999); Gottardi et al., Curr. Biol. 11:R792-4 (2001); Huber et al., Cell 105:391-402 (2001). β-catenin has been identified as both a mediator of cell adhesion at the plasma membrane and as a transcriptional activator.

The term “CBP protein” refers to the protein that is also known as CREB-binding protein, where CREB is an abbreviation for “cAMP-response element binding.” This protein is well known in the art, see, e.g., Takemaru et al., J. Cell Biol. 149:249-54 (2000) and U.S. Pat. No. 6,063,583.

The term “p300 protein” refers to a protein that is well known in the art. See, e.g., Gusterson, R. J. et al., J Biol Chem. 2003 Feb. 28; 278(9):6838-47; An and Roeder, J Biol Chem. 2003 Jan. 17; 278(3):1504-10; Rebel, V. I. et al., Proc Natl Acad Sci USA. 2002 Nov. 12; 99(23): 14789-94; and U.S. Pat. No. 5,658,784, as well as references cited therein.

The phrase “likelihood that a cell will differentiate rather than proliferate” refers to the probability of a cell that will differentiate rather than proliferate. Such a probability may be expressed and/or measured by the ratio of the number of cells that differentiate to that of cells that proliferate under given conditions. An agent that “increases the likelihood that a cell will differentiate rather than proliferate” refers to a compound that increases the ratio of the number of cells that differentiate to that of cells that proliferate when the compound is present compared to the same ratio when the compound is absent. Likewise, an agent that “increases the likelihood that a cell will proliferate rather than differentiate” refers to a compound that increases the ratio of the number of cell that proliferate to that of cells that differentiate when the compound is present compared to the same ratio when the compound is absent.

The phrase “Wnt pathway” refers to a signaling cascade that may be initiated by the binding of Wnt proteins (secreted glycoproteins) to frizzled seven-transmembrane-span receptors. This pathway is known and characterized in the art and is the subject of numerous articles and reviews (see, e.g., Huelsken and Behrens, J. Cell Sci. 115: 3977-8, 2002; Wodarz et al., Annu. Rev. Cell Dev. Biol. 14:59-88 (1998); Morin, P. J., Bioessays 21:1021-30 (1999); Moon et al., Science 296:1644-46 (2002); Oving et al., Eur. J. Clin. Invest. 32:448-57 (2002); Sakanaka et al., Recent Prog. Horm. Res. 55: 225-36, 2000).

Agents

In one aspect the present invention provides agents that may be used in the methods described above. Agents useful in the methods of the present invention may be identified by screening compounds of formula (I):

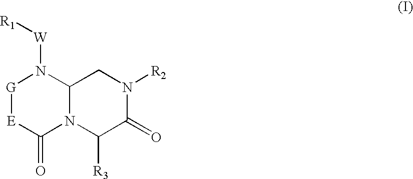

wherein:

E is -(ZR₄)- or —(C═O)—;

G is nothing, —(XR₅)—, or —(C═O)—;

W is —Y(C═O)—, —(C═O)NH—, —(SO₂)— or nothing;

Y is oxygen or sulfur;

X or Z is independently nitrogen or CH;

R₁, R₂, R₃, R₄, and R₅ are the same or different and independently selected from the group consisting of:

an amino acid side chain moiety;

C₁₋₁₂ alkyl or substituted C₁₋₁₂ alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;

C₁₋₆alkoxy;

C₆₋₁₂aryl or substituted C₆₋₁₂aryl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl;

monocyclic aryl-alkyl having 5 to 7 ring members, which may have 1 to 2 heteroatoms selected from nitrogen, oxygen or sulfur, or substituted monocyclic aryl-alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

bicyclic aryl-alkyl having 8 to 10 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;

tricyclic aryl-alkyl having 5 to 14 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;

arylC₁₋₄alkyl or substituted arylC₁₋₄alkyl having one or more substituents independently selected from amino, amidino, guanidino, hydrazino, C₁₋₄alkylamino, C₁₋₄dialkylamino, C₃₋₆cycloalkyl, halogen, perfluoroC₁₋₄alkyl, C₁₋₆alkyl, C₁₋₃alkoxy, nitro, carboxyl, cyano, sulfuryl, hydroxyl, amide, C₁₋₆alkyloxyC₁₋₆acyl and morphorlinylC₁₋₆alkyl;

cycloalkylalkyl or substituted cycloalkylalkyl having one or more substituents independently selected from amino, amidino, guanidino, hydrazino, C₁₋₄alkylamino, C₁₋₄ dialkylamino, halogen, perfluoro C₁₋₄alkyl, C₁₋₄alkyl, C₁₋₃alkoxy, nitro, carboxyl, cyano, sulfuryl and hydroxyl; and

cycloalkyl or substituted cycloalkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl.

In certain embodiments, R₁, R₂, R₃, R₄, and R₅ are the same or different and independently selected from the group consisting of:

C₁₋₁₂ alkyl or substituted C₁₋₁₂ alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;

C₁₋₆alkoxy;

cycloalkylC₁₋₃alkyl;

cycloalkyl;

phenyl or substituted phenyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;

phenylC₂₋₄alkyl or phenylC₂₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, sulfide, hydroxyl;

naphthyl or substituted naphthyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;

naphthylC₁₋₄alkyl or naphthylC₁₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;

benzyl or substituted benzyl having one or more substituents independently selected from amino, amidino, guanidino, hydrazino, C₁₋₄alkylamino, C₁₋₄dialkylamino, halogen, perfluoro C₁₋₄alkyl, trifluoroC₁₋₄alkyl, C₁₋₆alkyl, C₁₋₃alkoxy, nitro, carboxyl, cyano, sulfuryl and hydroxyl;

bisphenylmethyl or substituted bisphenylmethyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

benzylphenyl amide, or substituted benzylphenyl amide having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

pyridyl or substituted pyridyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

pyridylC₁₋₄alkyl, or substituted pyridylC₁₋₄alkyl having one or more substituents independently selected from amino, amidino, guanidino, hydrazino, C₁₋₄alkylamino, C₁₋₄ dialkylamino, halogen, perfluoro C₁₋₄alkyl, C₁₋₄alkyl, C₁₋₃alkoxy, nitro, carboxyl, cyano, sulfuryl and hydroxyl;

pyrimidylC₁₋₄alkyl, or substituted pyrimidylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;

triazin-2-ylC₁₋₄alkyl, or substituted triazin-2-ylC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

imidazolylC₁₋₄alkyl or substituted imidazolylC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

benzothiazolinC₁₋₄alkyl or substituted benzothiazolinC₁₋₄alkyl having one or more substituents independently selected from amino, amidino, guanidino, hydrazino, C₁₋₄ alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;

phenoxazinC₁₋₄alkyl;

benzyl p-tolyl ether;

phenoxybenzyl;

N-amidinopiperazinyl-N—C₁₋₄alkyl;

quinolineC₁₋₄alkyl;

N-amidinopiperazinyl;

N-amidinopiperidinylC₁₋₄alkyl;

4-aminocyclohexylC₁₋₂alkyl; and

4-aminocyclohexyl.

As used herein, the term “amino acid side chain moiety” represents any amino acid side chain moiety present in naturally occurring proteins including (but not limited to) the naturally occurring amino aid side chain moieties identified in Table 1. Other naturally occurring amino acid side chain moieties of this invention include (but are not limited to) the side chain moieties of 3,5-dibromotyrosine, 3,5-diiodotyrosine, hydroxylysine, γ-carboxyglutamate, phosphotyrosine and phosphoserine. In addition, glycosylated amino acid side chains may also be used in the practice of this invention, including (but not limited to) glycosylated threonine, serine and asparagine.

Table 1

TABLE 1

| Amino Acid Side Chain Moiety | Amino Acid |
|---|---|
| —H | Glycine |
| —CH₃ | Alanine |
| —CH(CH₃)₂ | Valine |
| —CH₂CH(CH₃)₂ | Leucine |
| —CH(CH₃)CH₂CH₃ | Isoleucine |
| —(CH₂)₄NH₃⁺ | Lysine |
| —(CH₂)₃NHC(NH₂)NH₂⁺ | Arginine |
| 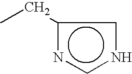 | Histidine |
| —CH₂COO⁻ | Aspartic acid |
| —CH₂CH₂COO⁻ | Glutamic acid |
| —CH₂CONH₂ | Asparagine |
| —CH₂CH₂CONH₂ | Glutamine |
| 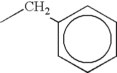 | Phenylalanine |
| 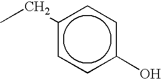 | Tyrosine |
| 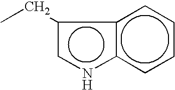 | Tryptophan |
| —CH₂SH | Cysteine |
| —CH₂CH₂SCH₃ | Methionine |
| —CH₂OH | Serine |
| —CH(OH)CH₃ | Threonine |
| 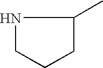 | Proline |
| 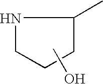 | Hydroxyproline |

In certain embodiments, E is -(ZR₄)- and G is —(XR₅)—, wherein Z is CH and X is nitrogen, and the compound has the following general formula (II):

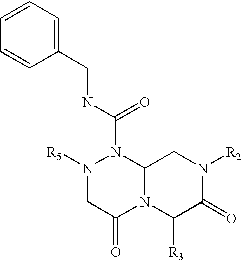

wherein R₂, R₃, and R₅ are as defined as in formula (I).

In certain embodiments, the compound has the following general formula (III):

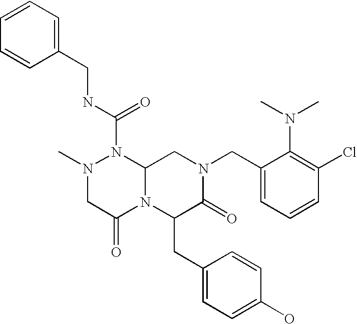

In certain embodiments, E is -(ZR₄)- and G is nothing, wherein Z is nitrogen, and the compound has the following general formula (IV):

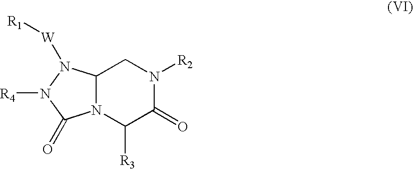

wherein R₁, R₂, R₃, R₄, and W are as defined in formula (I).

In certain embodiments, E is -(ZR₄)- and G is —(XR₅)—, wherein Z and X are independently CH, and the compound has a structure of Formula (V):

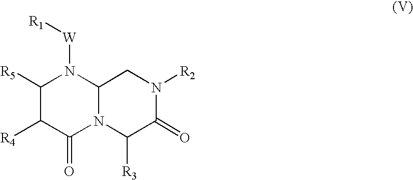

wherein R₁, R₂, R₃, R₄, R₅, and W are as defined in formula (I).

In certain embodiments, the compound has the following general formula (VI):

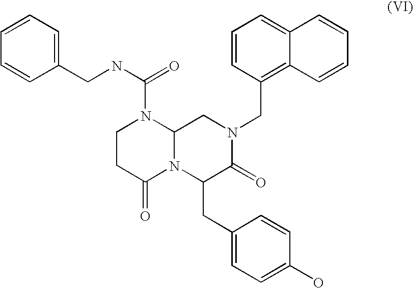

These compounds may be prepared by utilizing appropriate starting component molecules (hereinafter referred to as “component pieces”). Briefly, in the synthesis of reverse-turn mimetic structures having formula (I), first and second component pieces are coupled to form a combined first-second intermediate, if necessary, third and/or fourth component pieces are coupled to form a combined third-fourth intermediate (or, if commercially available, a single third intermediate may be used), the combined first-second intermediate and third-fourth intermediate (or third intermediate) are then coupled to provide a first-second-third-fourth intermediate (or first-second-third intermediate) which is cyclized to yield the reverse-turn mimetic structures of this invention. Alternatively, the reverse-turn mimetic structures of formula (I) may be prepared by sequential coupling of the individual component pieces either stepwise in solution or by solid phase synthesis as commonly practiced in solid phase peptide synthesis.

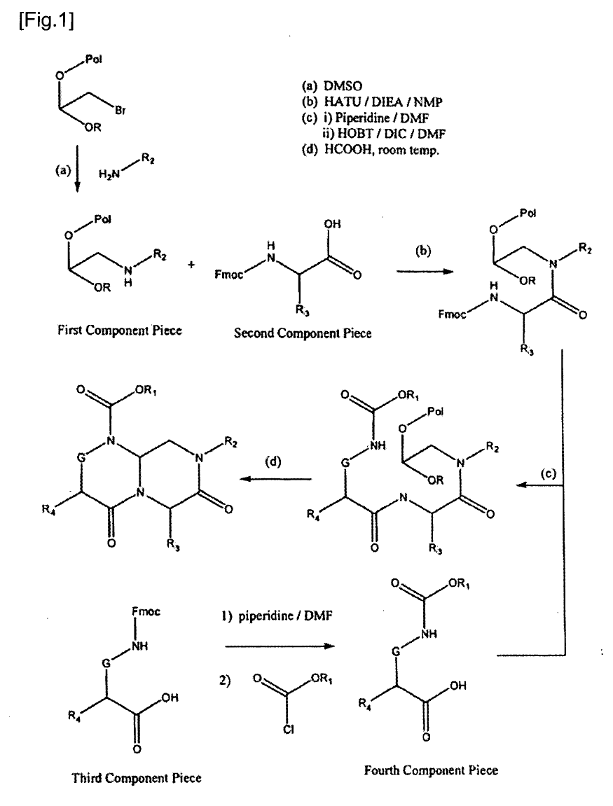

Specific component pieces and the assembly thereof to prepare compounds of the present invention are illustrated in FIG. 1. For example, a “first component piece” may have the following formula S1:

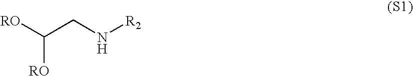

wherein R₂ is as defined above, and R is a protective group suitable for use in peptide synthesis, where this protection group may be joined to a polymeric support to enable solid-phase synthesis. Suitable R groups include alkyl groups and, in a preferred embodiment, R is a methyl group. In FIG. 1, one of the R groups is a polymeric (solid) support, indicated by “Pol” in the Figure. Such first component pieces may be readily synthesized by reductive amination of H₂N—R₂ with CH(OR)₂—CHO, or by a displacement reaction between H₂N—R₂ and CH(OR)₂—CH₂-LG (wherein LG refers to a leaving group, e.g., a halogen (Hal) group).

A “second component piece” may have the following formula S2:

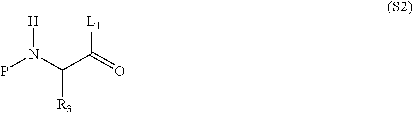

where P is an amino protection group suitable for use in peptide synthesis, L₁ is hydroxyl or a carboxyl-activation group, and R₃ is as defined above. Preferred protection groups include t-butyl dimethylsilyl (TBDMS), t-butyloxycarbonyl (BOC), methyloxycarbonyl (MOC), 9H-fluorenylmethyloxycarbonyl (FMOC), and allyloxycarbonyl (Alloc). N-Protected amino acids are commercially available; for example, FMOC amino acids are available from a variety of sources. In order for the second component piece to be reactive with the first component piece, L₁ is a carboxyl-activation group, and the conversion of carboxyl groups to activated carboxyl groups may be readily achieved by methods known in the art for the activation of carboxyl groups. Suitable activated carboxylic acid groups include acid halides where L₁ is a halide such as chloride or bromide, acid anhydrides where L₁ is an acyl group such as acetyl, reactive esters such as N-hydroxysuccinimide esters and pentafluorophenyl esters, and other activated intermediates such as the active intermediate formed in a coupling reaction using a carbodiimide such as dicyclohexylcarbodiimide (DCC). Accordingly, commercially available N-protected amino acids may be converted to carboxylic activated forms by means known to one of skill in the art.

In the case of the azido derivative of an amino acid serving as the second component piece, such compounds may be prepared from the corresponding amino acid by the reaction disclosed by Zaloom et al. (J. Org. Chem. 46:5173-76, 1981).

Alternatively, the first component piece of the invention may have the following formula S1′:

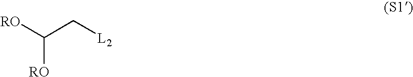

wherein R is as defined above and L₂ is a leaving group such as halogen atom or tosyl group, and the second component piece of the invention may have the following formula S2′:

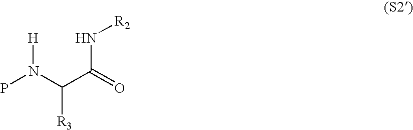

wherein R₂, R₃ and P are as defined above,

A “third component piece” of this invention may have the following formula S3:

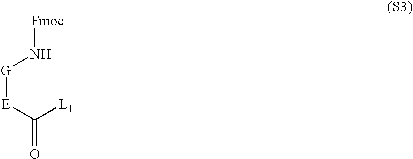

where G, E, L₁ and L₂ are as defined above. Suitable third component pieces are commercially available from a variety of sources or can be prepared by methods well known in organic chemistry.

Thus, as illustrated above, the reverse-turn mimetic compounds of formula (I) may be synthesized by reacting a first component piece with a second component piece to yield a combined first-second intermediate, followed by reacting the combined first-second intermediate with the third-fourth intermediate (or the third component piece) to provide a combined first-second-third-fourth intermediate (or a combined first-second-third intermediate), and then cyclizing this intermediate to yield the reverse-turn mimetic structure.

Methods of Use

The present invention provides compounds of formula (a) that inhibit a subset of catenin/TCF induced transcription.

In another aspect, the present invention provides a method for selectively inhibiting expression of genes targeted by the WNT/β-catenin pathway, the method comprising administering a compound to a composition, the composition comprising genes targeted by the WNT/β-catenin pathway, the compound causing a change in expression of the genes targeted by the WNT/β-catenin pathway.

In another aspect, the present invention provides a method for inducing the differentiation of stem cells comprising contacting the stem cell with an agent that induces cell differentiation into a specific lineage such as, e.g., osteoblast, osteocyte, cardiomyocyte, skeletal muscle cell, blood cell, neuron, and pancreatic beta cell.

Cell proliferation and cell differentiation may be characterized with any appropriate methods known in the art. Such methods include flow cytometric analysis, real time RT-PCR and cell proliferation assay as described in the examples.

In another aspect, the present invention provides a method for maintaining a stem cell in an undifferentiated state, comprising contacting the stem cell with an agent that inhibits cell differentiation or promotes cell proliferation in an amount effective to maintain the stem cell in an undifferentiated state.

Stem cell therapy offers an opportunity to treat many degenerative diseases caused by the premature death of malfunction of specific cell types and the body's failure to replace or restore them. Possible therapeutic uses of stem cells include immunological conditioning of patients for organ transplants, treatment of autoimmune diseases such as muscular dystrophy, multiple sclerosis and rheumatoid arthritis, repair of damaged tissues such as stroke, spinal injury and burn, treatment of neurodegenerative disease like Lou Gehrig's disease, and neurological conditions such as Parkinson's Huntington's and Alzheimer's diseases, treatment of leukaemia, sickle cell anemia, heart disease, and diabetes. For most stem cell therapy, embryonic stem cells or adult stem cells may be cultured in vitro, induced to differentiate to the desired cell type and transplant to a patient. For successful culture of stem cells, stem cells need to be maintained in an undifferentiated condition.

To maintain stem cells in an undifferentiated condition, compounds according to the present invention, such as those that promote cell proliferation or inhibit cell differentiation, may be used at various stages of stem cell culture. For instance, such a compound may be used when the stem cells are isolated from their source tissue. Alternatively, it may be added to culture media after certain period of culture. It may also be continuously present in culture media to maintain the stem cells in an undifferentiated state. The concentration of the compound may be optimized by adjusting the amount of the compound to the level at which stem cells are maintained in an undifferentiated state, or the differentiation of stem cells is reduced compared to the stem cells cultured in the absence of the compounds, and other aspects of the cell culture (e.g., cell viability rate and cell proliferation rate) is not adversely affected.

These and other methods of the present invention may be practiced with a chemical agent, such as a chemical agent identified herein as COMPOUND A-F as well as its analogs.

Pharmaceutical Compositions and Administration

The compounds according to the present invention can be incorporated into pharmaceutical compositions suitable for administration. Such compositions typically comprise the compound and a pharmaceutically acceptable carrier. “Pharmaceutically acceptable carrier” refers to solvents, dispersion media, coatings, antibacterial and antifungal agents, isotonic and absorption delaying agents and the like, compatible with pharmaceutical administration. The use of such media and agents for pharmaceutically active substances is well known in the art. Supplementary active compound can also be incorporated into the compositions.

The pharmaceutical composition of the present invention may be administered parenterally, topically, orally, or locally for therapeutic treatment. A variety of aqueous carriers may be used, e.g., water, buffered water, 0.4% saline, 0.3% glycine and the like, and may include other proteins for enhancing stability, such as albumin, lipoprotein, globulin, etc. The resulting composition may be sterilized by conventional, well-known sterilization techniques. The solutions may be packaged for use or filtered under aseptic conditions and lyophilized, the lyophilized preparation being combined with a sterile solution prior to administration.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder (e.g., microcrystalline cellulose, gum tragacanth or gelatin); an excipient (e.g., starch or lactose), a disintegrating agent (e.g., alginic acid, Primogel, or corn starch); a lubricant (e.g., magnesium stearate or Sterotes); a glidant (e.g., colloidal silicon dioxide); a sweetening agent (e.g., sucrose or saccharin); a flavoring agent (e.g., peppermint, methyl salicylate, or orange flavoring).

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC 50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

TABLE 2
Bioactivity test of compounds selected from the compounds of the present
invention and % change values thereof, which are measured by the Osteogenesis assay
and Cardiomyogenesis assay as described in Examples 2 and 3.

| Serial | | | | |
|---|---|---|---|---|
| No. | Structure | M. W. | Osteogenesis | Cardiomyogenesis |
| #1 | 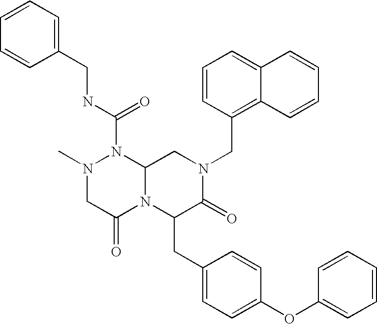 | 639.75 | 0.36 | 1.21 |
| #2 | 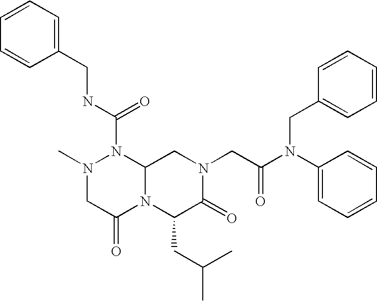 | 596.72 | 1.09 | 1.11 |
| #3 | 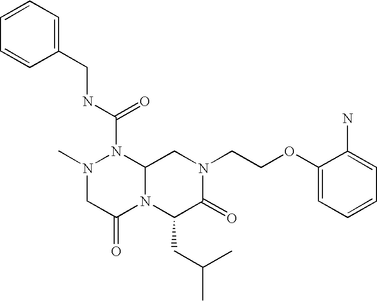 | 508.61 | 1.29 | 1.25 |
| #4 | 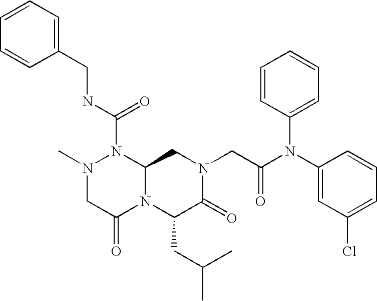 | 617.14 | 0.59 | 0.40 |
| #5 | 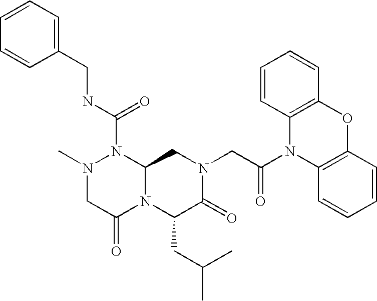 | 582.70 | 0.29 | 0.36 |
| #6 | 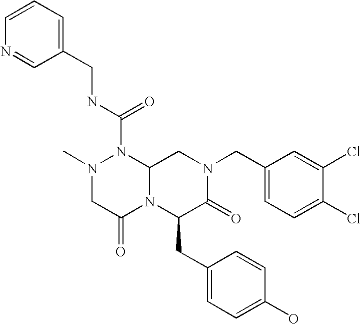 | 583.47 | 0.51 | 0.47 |
| #7 | 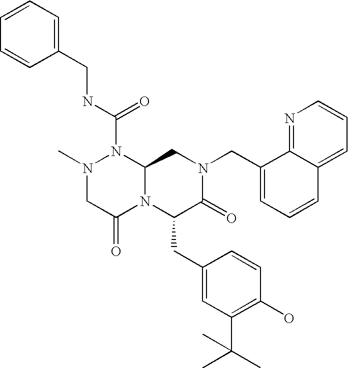 | 620.75 | 0.51 | 1.22 |
| #8 | 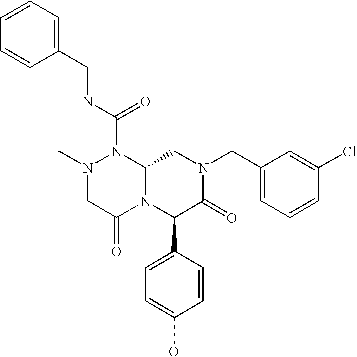 | 534.01 | 0.55 | 0.65 |
| #9 | 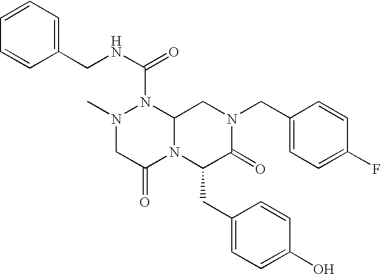 | 531.59 | 0.24 | 1.59 |
| #10 | 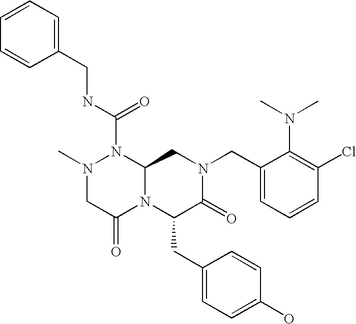 | 591.11 | 0.23 | 0.26 |
| #11 | 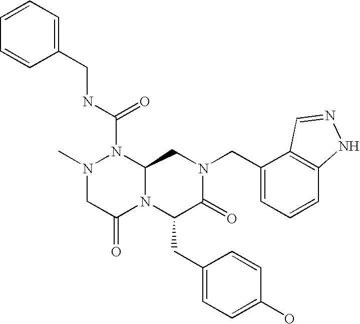 | 553.62 | 0.30 | 0.07 |
| #12 | 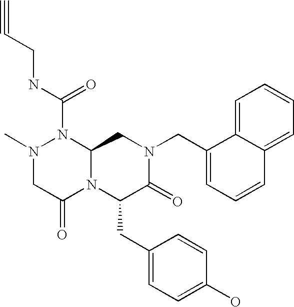 | 511.58 | 0.76 | 1.27 |
| #13 | 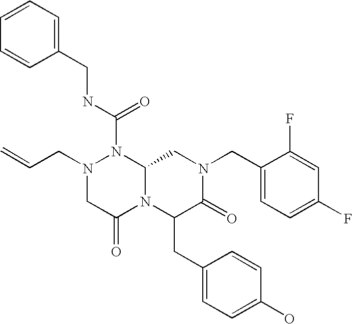 | 575.61 | 0.47 | 1.02 |
| #14 | 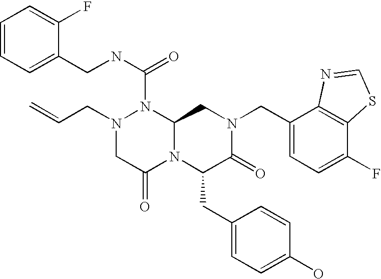 | 632.69 | 0.29 | 0.00 |
| #15 | 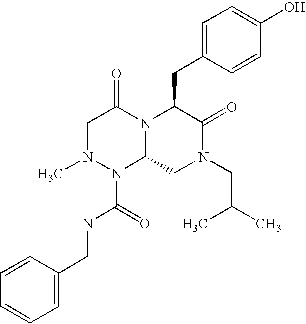 | 479.58 | 1.12 | 0.41 |
| #16 | 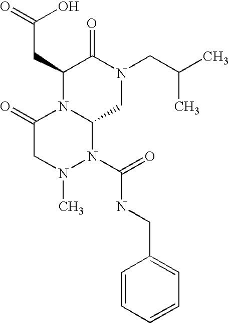 | 431.49 | 0.97 | 1.42 |
| #17 | 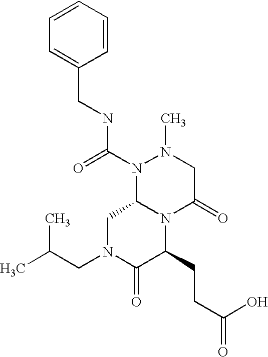 | 445.52 | 1.29 | 2.52 |
| #18 | 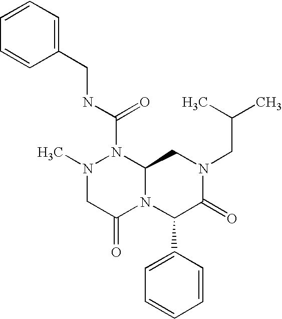 | 449.55 | 0.93 | 2.01 |
| #19 | 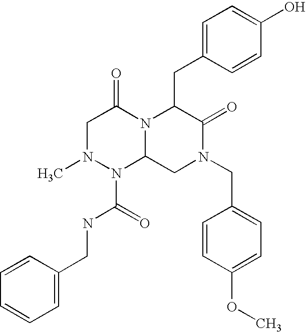 | 543.62 | 0.70 | 3.03 |
| #20 | 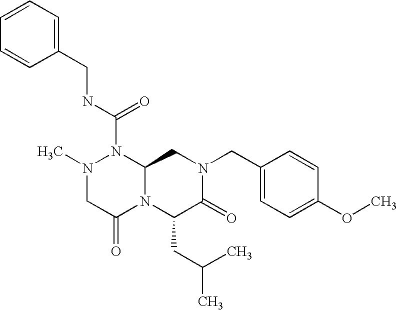 | 493.60 | 1.06 | 2.17 |
| #21 | 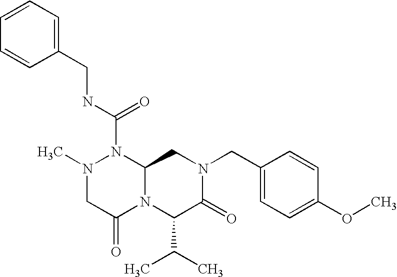 | 479.58 | 1.21 | 0.62 |
| #22 | 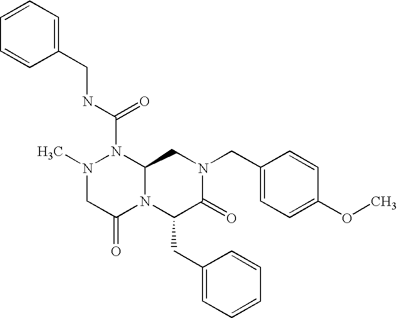 | 527.62 | 0.70 | 1.16 |
| #23 | 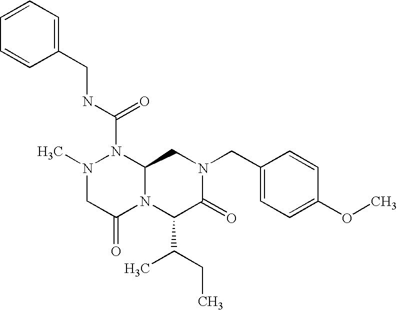 | 493.60 | 0.86 | 1.69 |
| #24 | 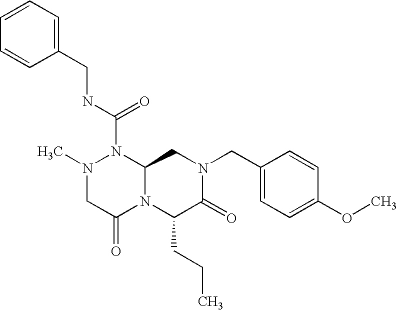 | 479.58 | 0.85 | 1.57 |
| #25 | 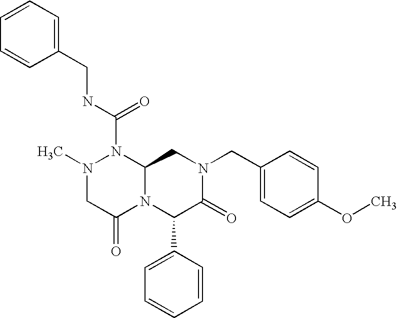 | 513.60 | 0.60 | 1.78 |
| #26 | 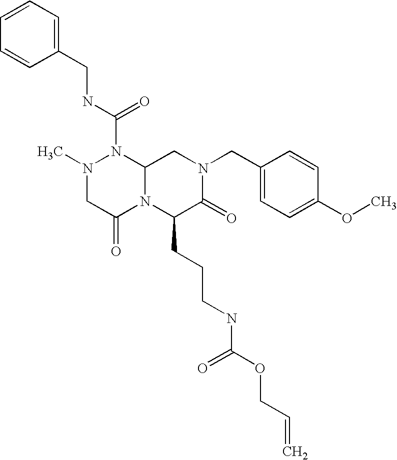 | 578.67 | 1.01 | 1.74 |
| #27 | 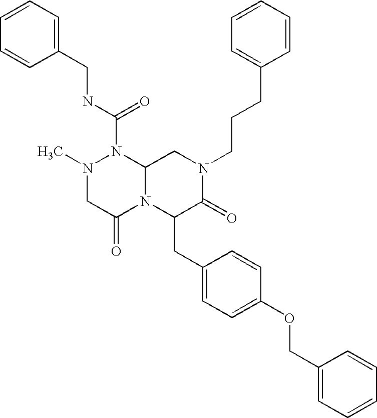 | 631.77 | 0.96 | 0.52 |
| #28 | 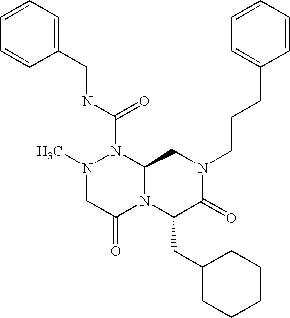 | 531.70 | 0.26 | 1.23 |
| #29 | 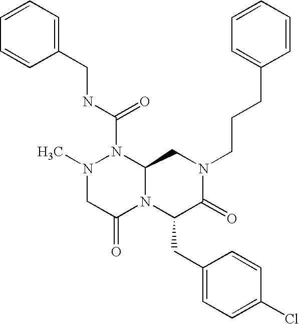 | 560.10 | 0.91 | 1.71 |
| #30 | 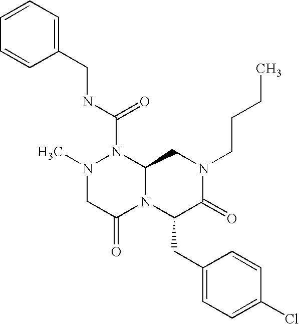 | 498.02 | 1.12 | 1.75 |
| #31 | 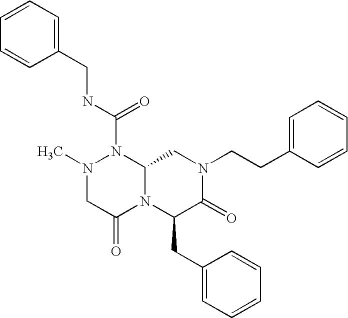 | 511.62 | 1.00 | 1.10 |
| #32 | 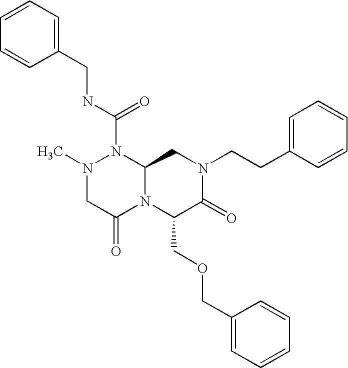 | 541.65 | 0.94 | 0.95 |
| #33 | 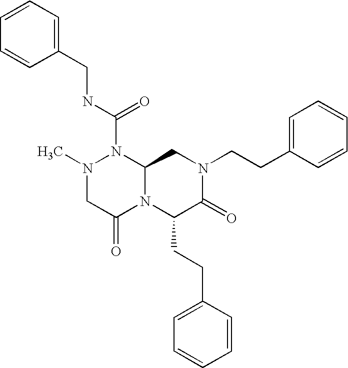 | 525.65 | 1.85 | 0.28 |
| #34 | 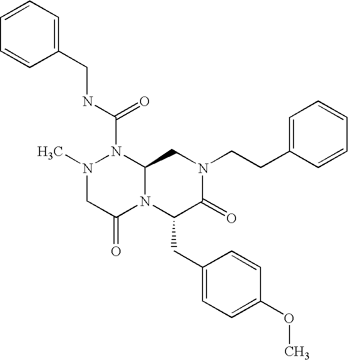 | 541.65 | 1.03 | 1.71 |
| #35 | 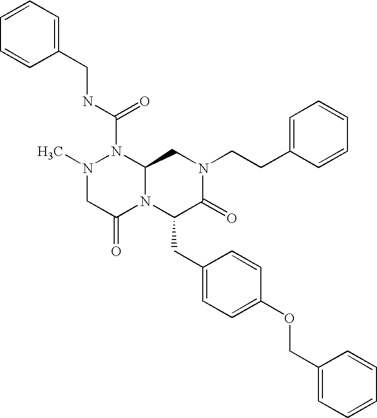 | 617.75 | 0.94 | 1.03 |
| #36 | 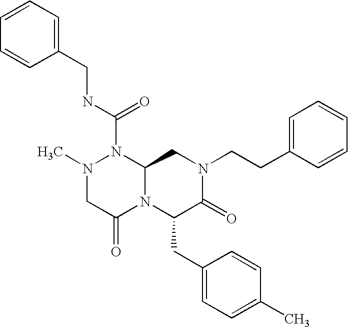 | 525.65 | 1.55 | 0.54 |
| #37 | 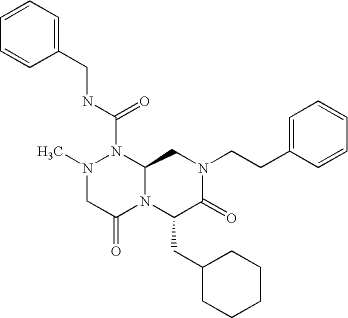 | 517.67 | 0.95 | 0.98 |
| #38 | 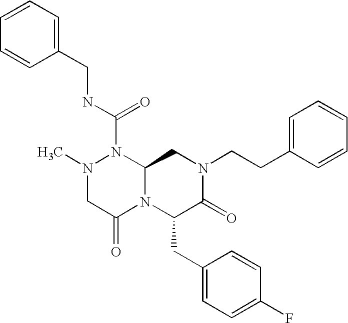 | 529.61 | 0.48 | 0.69 |
| #39 | 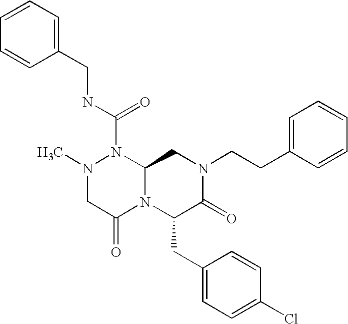 | 546.07 | 0.79 | 0.29 |
| #40 | 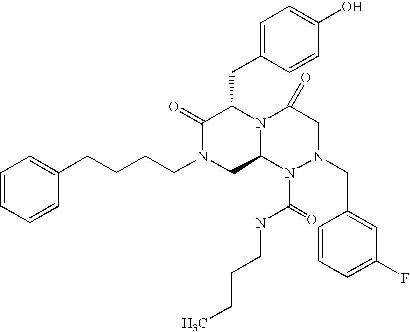 | 615.75 | 0.69 | 1.11 |
| #41 | 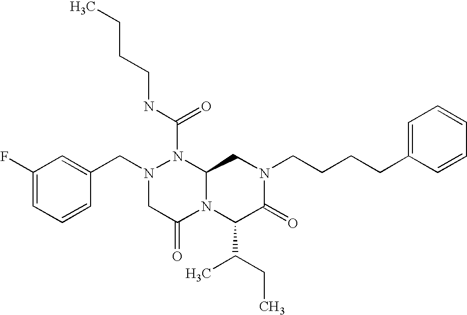 | 565.73 | 0.60 | 2.15 |
| #42 | 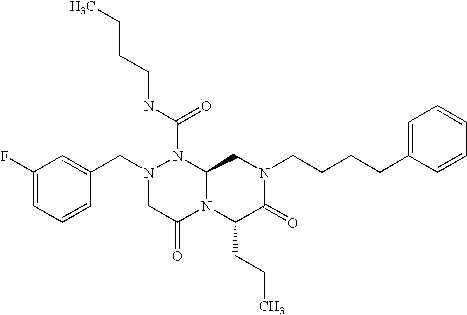 | 551.70 | 0.31 | 1.87 |
| #43 | 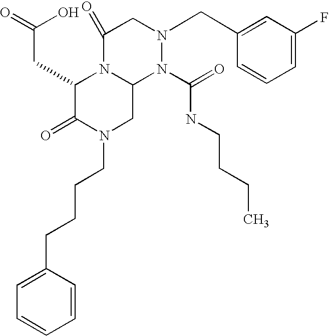 | 567.66 | 0.33 | 1.17 |
| #44 | 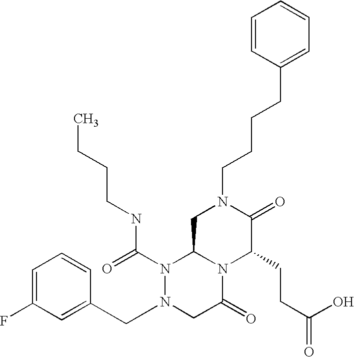 | 581.69 | 0.56 | 1.98 |
| #45 | 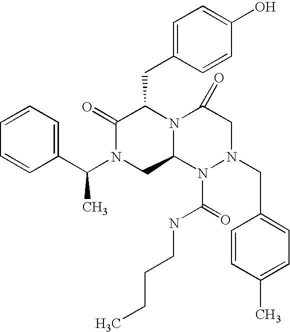 | 583.73 | 1.08 | 0.00 |
| #46 | 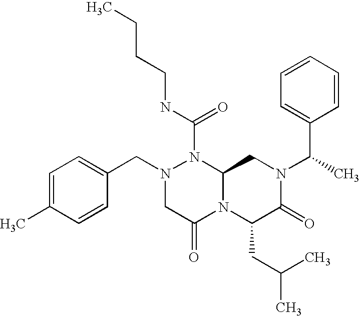 | 533.71 | 1.02 | 1.90 |
| #47 | 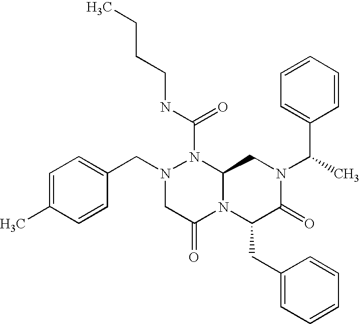 | 567.73 | | 2.32 |
| #48 | 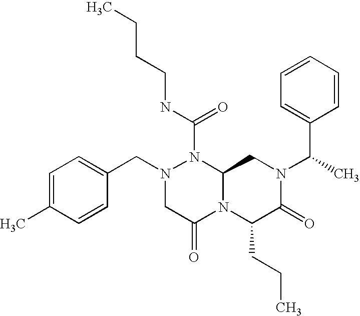 | 519.69 | | 1.02 |
| #49 | 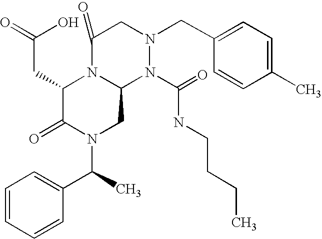 | 535.64 | | 1.14 |
| #50 | 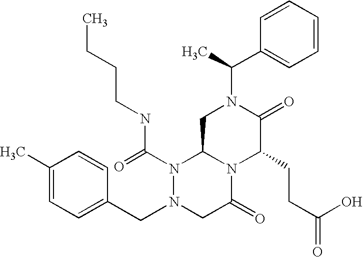 | 549.67 | | 0.26 |
| #51 | 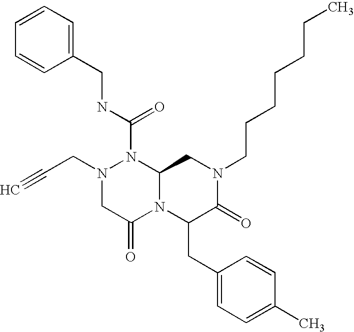 | 543.71 | 0.31 | 0.49 |
| #52 | 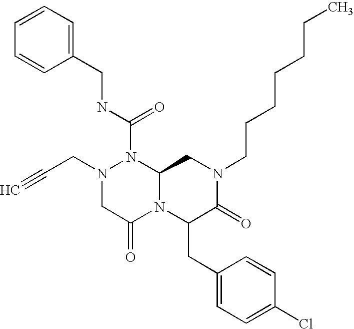 | 564.13 | | 1.99 |
| #53 | 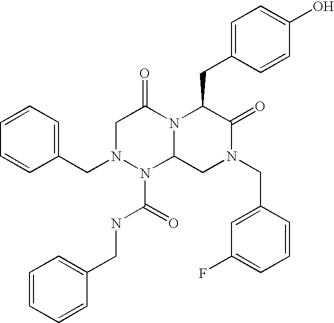 | 607.68 | | 1.95 |
| #54 | 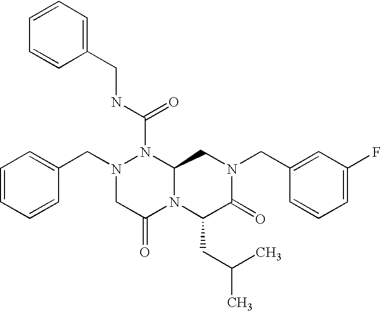 | 557.67 | | 1.42 |
| #55 | 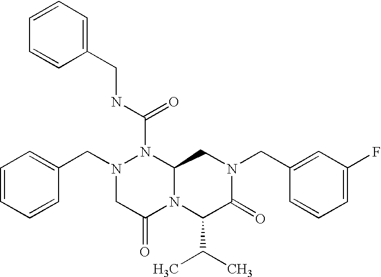 | 543.64 | 0.98 | 1.26 |
| #56 | 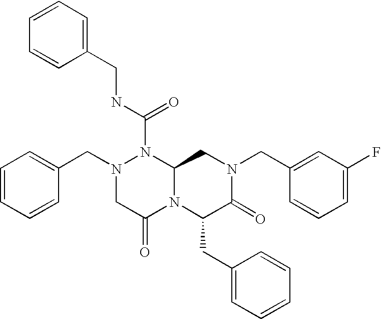 | 591.68 | | 1.08 |
| #57 | 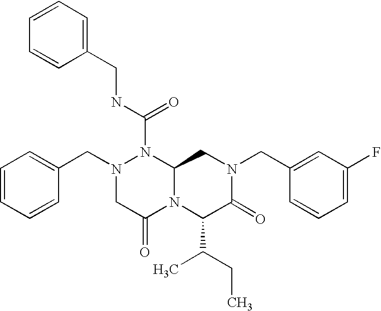 | 557.67 | 0.64 | 0.43 |
| #58 | 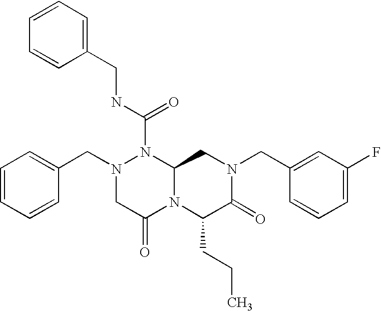 | 543.64 | | 1.91 |
| #59 | 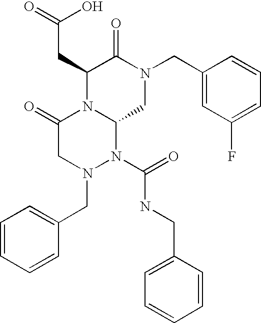 | 559.60 | | 0.48 |
| #60 | 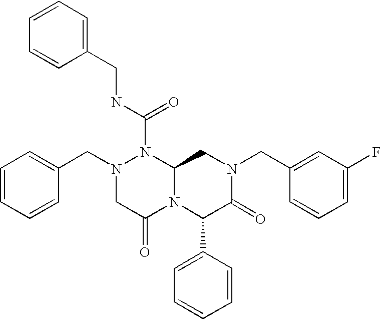 | 577.66 | | 1.23 |
| #61 | 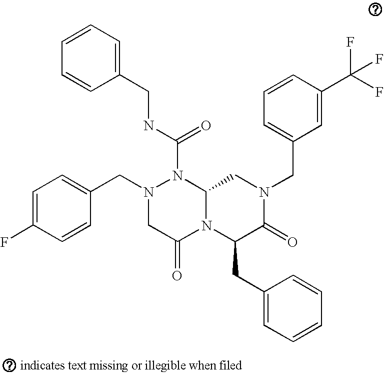 | 659.68 | | 1.06 |
| #62 | 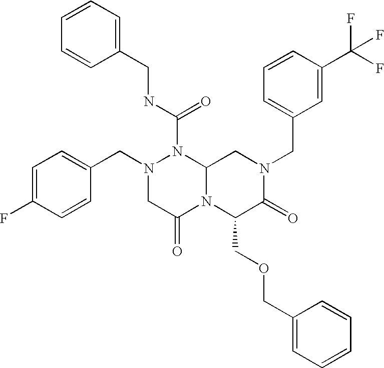 | 689.71 | | 0.89 |
| #63 | 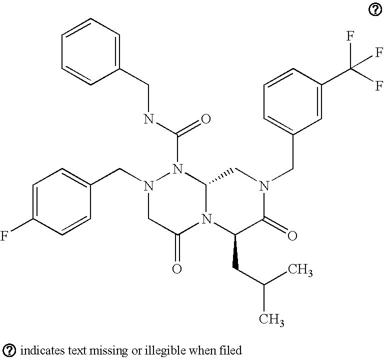 | 625.66 | 0.41 | 0.47 |
| #64 | 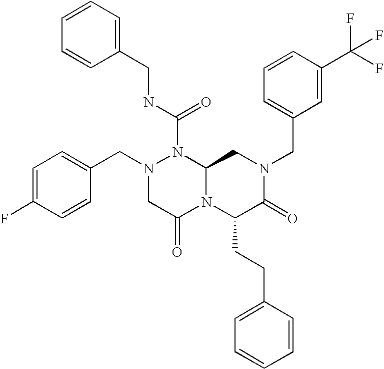 | 673.71 | 0.79 | 2.83 |
| #65 | 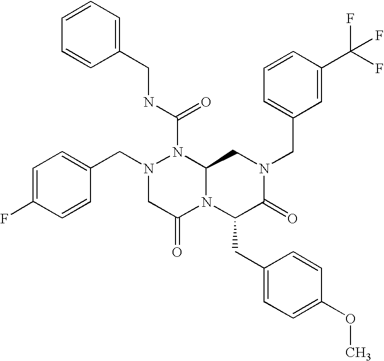 | 689.71 | 1.34 | 2.54 |
| #66 | 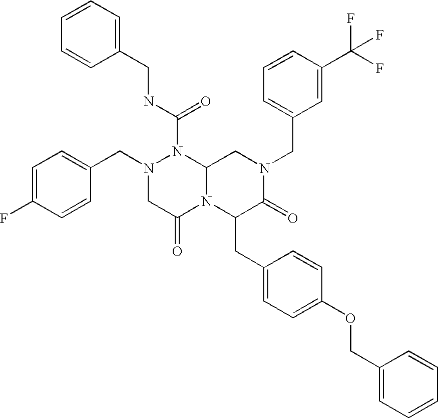 | 765.80 | 2.02 | 3.13 |
| #67 | 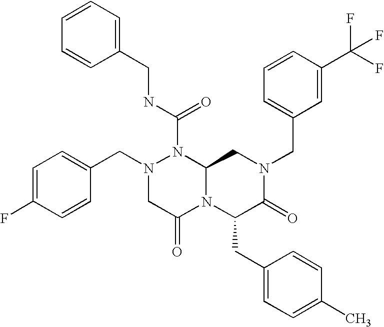 | 673.71 | 0.80 | 1.50 |
| #68 | 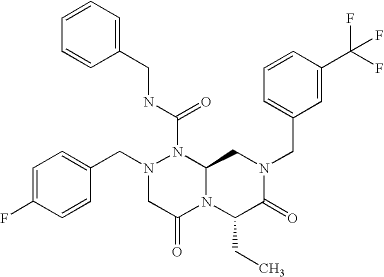 | 597.61 | 0.91 | 0.69 |
| #69 | 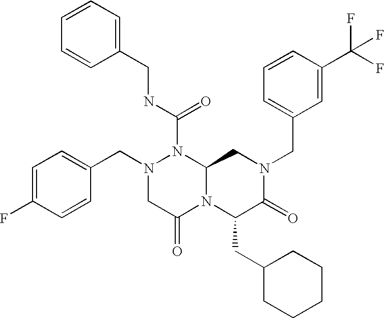 | 665.73 | 0.71 | 1.69 |
| #70 | 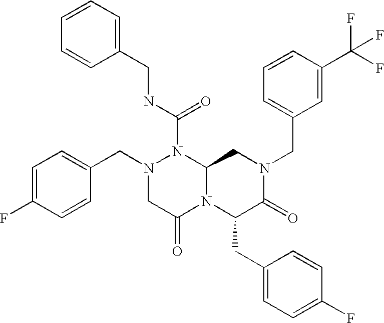 | 677.67 | 1.03 | 0.92 |
| #71 | 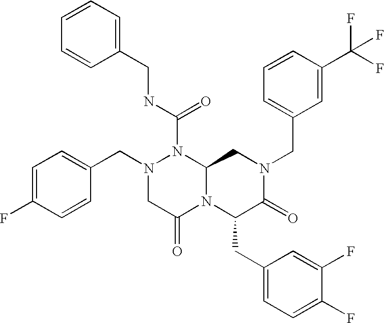 | 695.66 | | 4.63 |
| #72 | 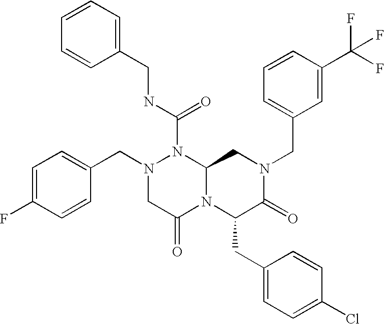 | 694.13 | 0.84 | 1.76 |
| #73 | 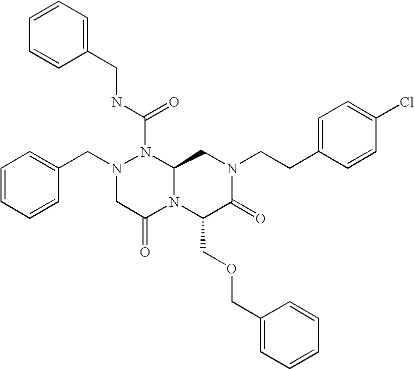 | 652.19 | 0.64 | 1.57 |
| #74 | 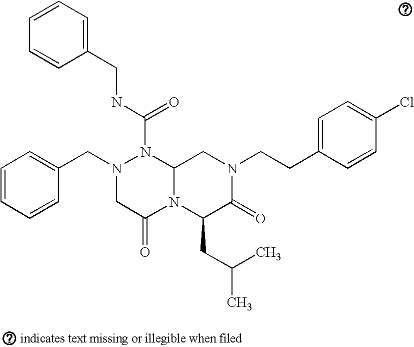 | 588.15 | 0.54 | 1.59 |
| #75 | 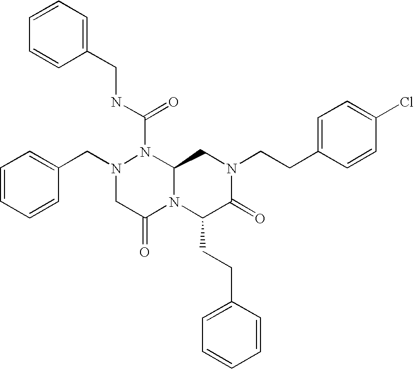 | 636.19 | 1.16 | 0.50 |
| #76 | 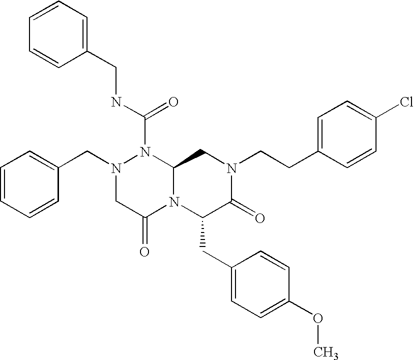 | 652.19 | 1.58 | 1.24 |
| #77 | 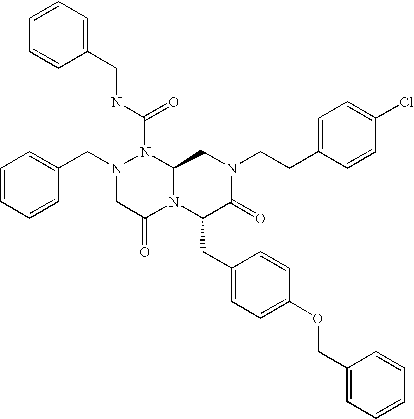 | 728.29 | 0.98 | 1.40 |
| #78 | 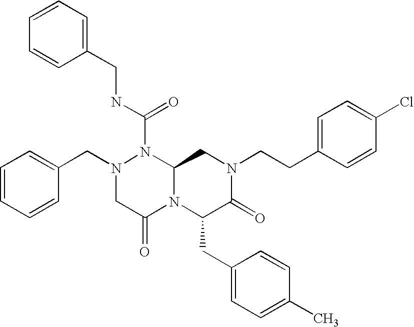 | 636.19 | 0.75 | 1.70 |
| #79 | 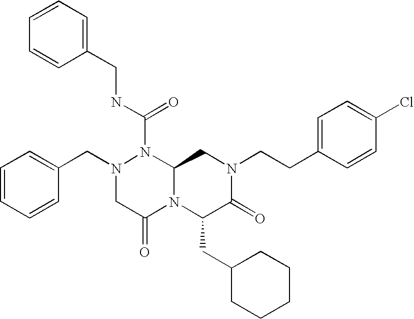 | 628.21 | 0.58 | 1.67 |
| #80 | 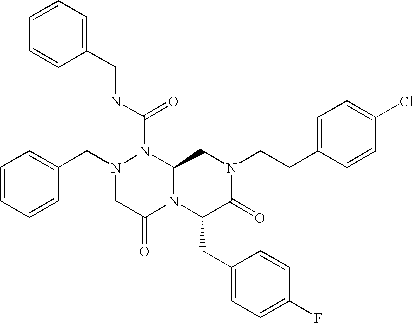 | 640.16 | 0.19 | 1.49 |
| #81 | 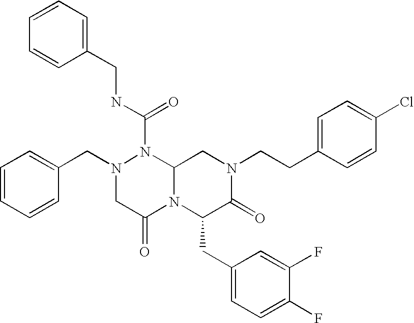 | 658.15 | 1.84 | 0.81 |
| #82 | 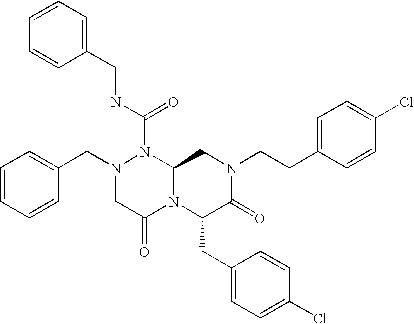 | 656.61 | 2.00 | 0.97 |
| #83 | 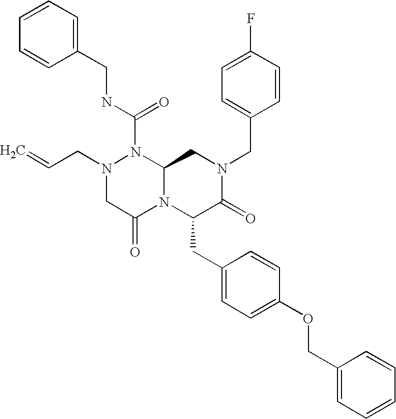 | 647.75 | 0.36 | 2.02 |
| #84 | 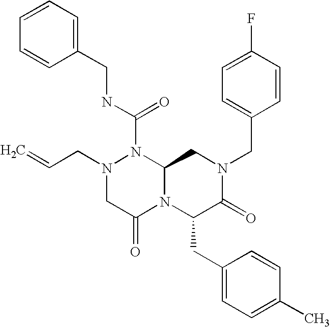 | 555.65 | 0.69 | 1.32 |
| #85 | 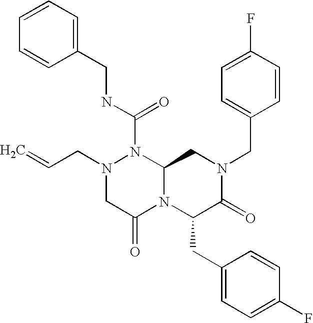 | 559.61 | 0.62 | 1.65 |
| #86 | 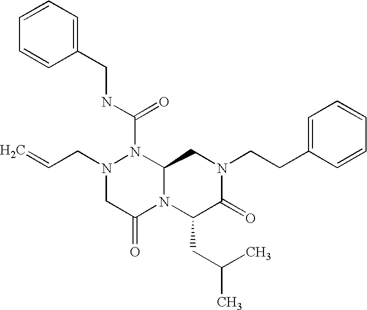 | 503.64 | 0.54 | 1.34 |
| #87 | 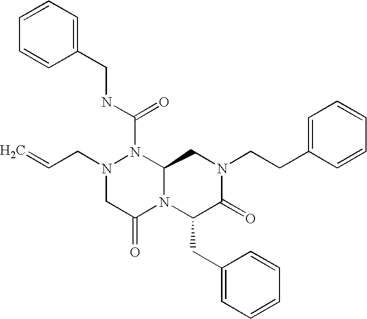 | 537.66 | 0.59 | 0.89 |
| #88 | 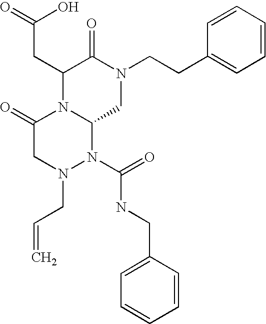 | 505.57 | 0.64 | 1.34 |
| #89 | 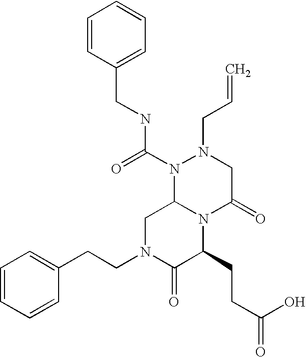 | 519.60 | 1.16 | 1.26 |
| #90 | 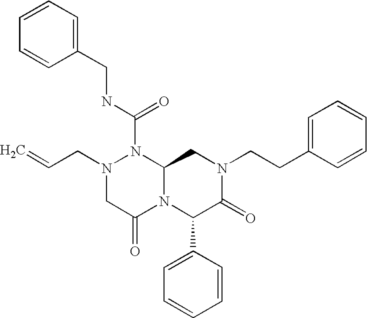 | 523.63 | 0.51 | 2.10 |
| #91 | 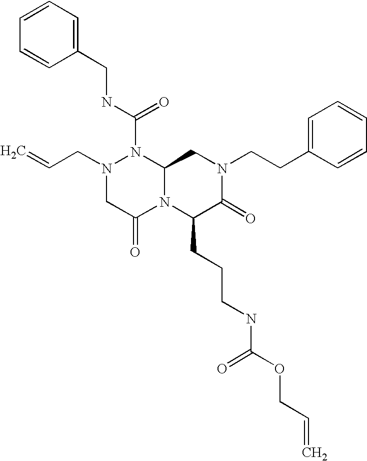 | 588.71 | 1.13 | 2.23 |
| #92 | 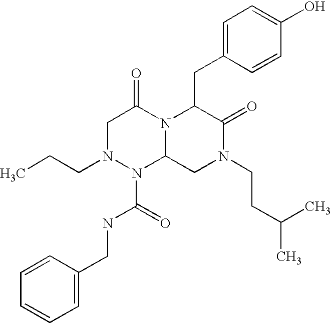 | 521.66 | 0.70 | 2.13 |
| #93 | 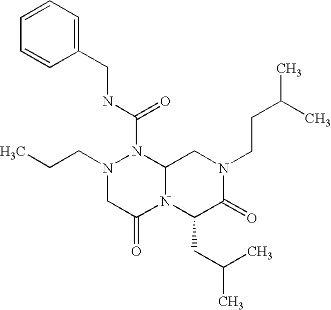 | 471.64 | 0.63 | 0.57 |
| #94 | 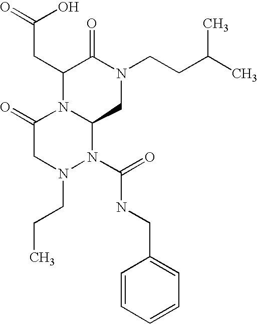 | 473.57 | 0.69 | 3.02 |
| #95 | 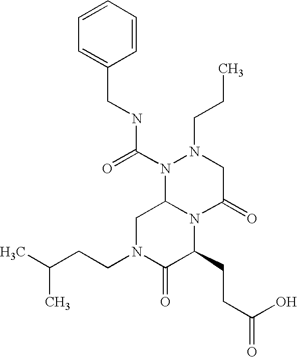 | 487.60 | 1.19 | 2.22 |
| #96 | 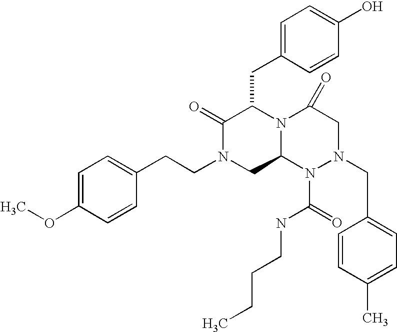 | 613.76 | 0.77 | 0.54 |
| #97 | 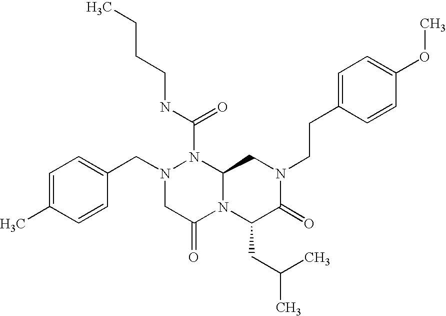 | 563.74 | 0.54 | 0.71 |
| #98 | 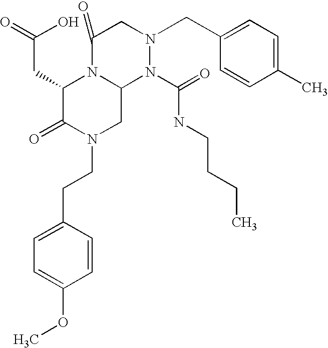 | 565.66 | 0.26 | 3.34 |
| #99 | 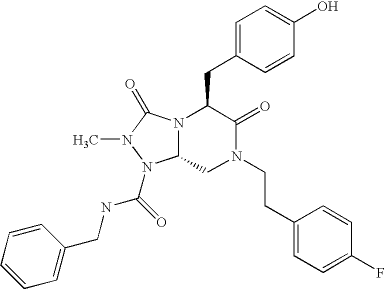 | 531.59 | 0.46 | 1.74 |
| #100 | 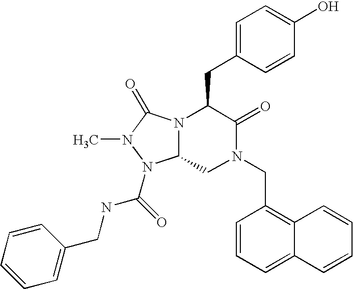 | 549.63 | 1.11 | 1.35 |
| #101 | 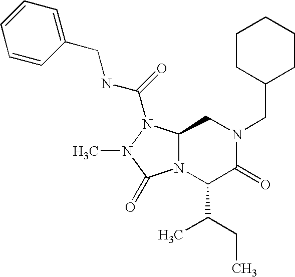 | 455.60 | 1.28 | 2.32 |
| #102 | 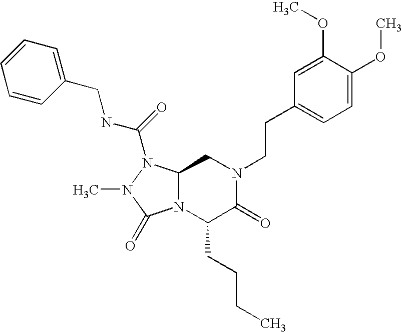 | 523.63 | 1.61 | 1.01 |
| #103 | 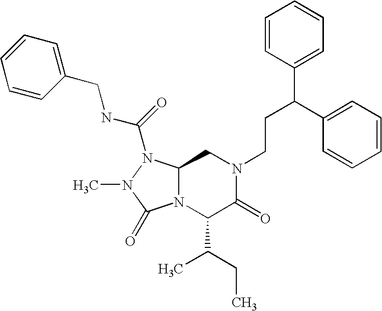 | 553.70 | 0.98 | 1.98 |
| #104 | 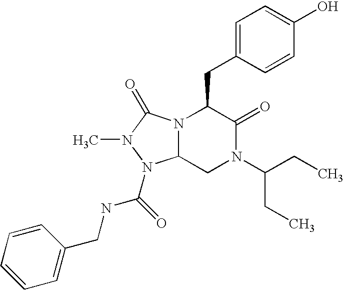 | 479.58 | 1.42 | 0.62 |
| #105 | 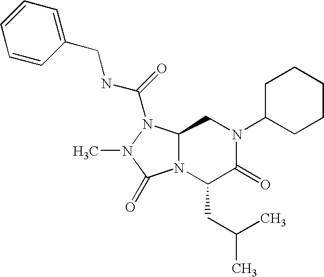 | 441.57 | 0.80 | 1.12 |
| #106 | 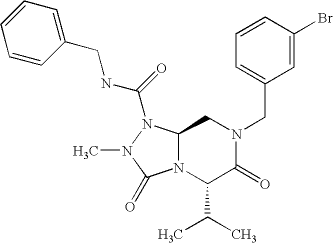 | 514.42 | 0.37 | 1.25 |
| #107 | 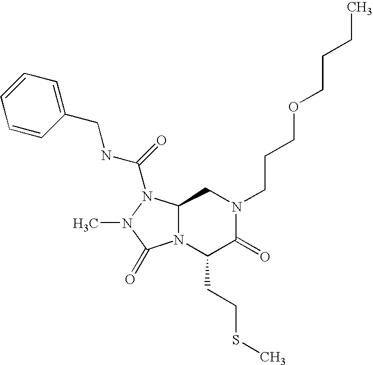 | 491.65 | 0.66 | 0.59 |
| #108 | 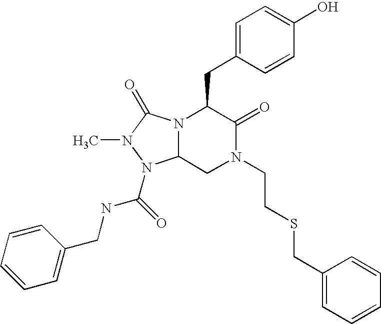 | 559.69 | 0.94 | 1.19 |
| #109 | 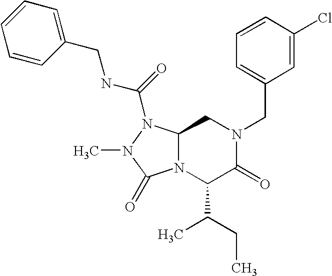 | 484.00 | 1.00 | 0.97 |
| #110 | 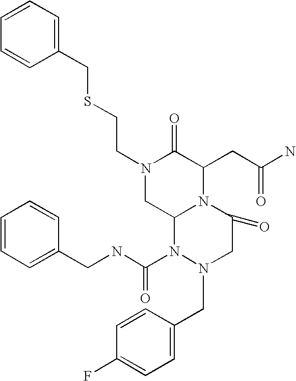 | 618.73 | 0.63 | 1.18 |
| #111 | 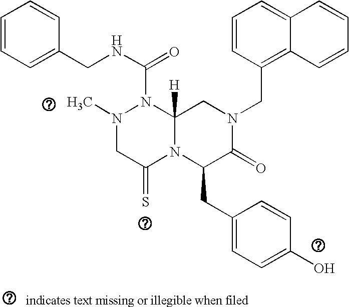 | 563.65 | | 0.10 |
| #112 | 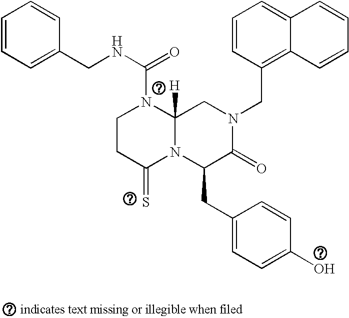 | 548.63 | | 0.95 |

The following examples are provided to illustrate the invention and are not to be construed as a limitation thereon.

### PREPARATION EXAMPLES

### Preparation Example 1

### Preparation of N-Benzylcarbamoyl-N′-methyl-hydrazino Acetic Acid

### (1) Preparation of N-Boc-N-Methyl Hydrazine

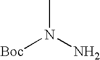

To a stirred suspension of methylhydrazine sulfate (10 g, 0.693 mol) in water (2.0 L) under ice-water external bath was carefully added NaHCO₃ to pH 11˜12. The reaction mixture was stirred vigorously and DiBoc (1.1 eq) in THF (2.0 L) was poured into the solution. After stirring at room temperature overnight, the organic layer was removed and extracted with ethyl acetate (1.0 L×3). The combined solution was dried over sodium sulfate and evaporated in vacuo to give the title compound (80 g, 79% as pale yellow oily compound), which was used in the next step without further purification.

### (2) Preparation of N-Boc-N-methyl-N′-Benzylcarbamoyl-hydrazine

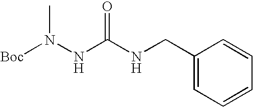

A 5.0 L, two-necked, round-bottom-flask was fitted with a glass stopper and a calcium chloride tube. To a stirred solution, in the flask, of N′-Boc-N′-Methyl Hydrazine (80 g, 0.547 mol) in THF (500 ml) under ice-water external bath were carefully added benzyl isocyanate (39 mL, 300 mmol) in THF (30 ml) via a dropping funnel. After 2 hrs, the solution was evaporated. The residue was slurried with Hexane (Using little amount ethyl acetate) to get a white solid. The colorless solid that separated was filtered and washed with minimum amount of hexane and dried under vacuum for overnight to give the title compound (124 g, 81% as white solid). ¹H NMR (CDCl₃, 300 MHz): δ1.41 (9H, s), δ3.10 (3H, s), δ4.45 (2H, d, J=6 Hz), δ5.71 (1H, t, J=6 Hz), δ7.04 (1H, br), δ7.28 (5H, m)

### (3) Preparation of N-methyl-N′-benzylcarbamoyl-hydrazine

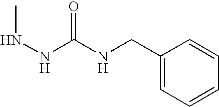

A 5 L, two-necked, round-bottom-flask was fitted with a glass stopper and reflux condenser connected to a calcium chloride tube. A solution of N-benzylcarbamoyl-N-Boc-N′-methylhydrazine (124 g, 0.443 mol) in 4M HCl (in Dioxane, 500 ml) was added in the flask and then 1,4-dioxane (3 L) was added with stirring. The reaction mixture was stirred at room temperature overnight. and evaporated in vacuo and NaHCO₃ aqueous solution was added slowly (pH 11˜12) under ice-water external bath. Aqueous layer was separated with Ethyl acetate (3 times) and dried over Na₂SO₄. The residue was evaporated in vacuo and slurried with hexane and EtOAc to get a white solid. The solid was filtered and washed with minimum amount of hexane and dried under vacuum for overnight to give the title compound (72 g, 91% as white solid).

### (4) Preparation t-Butyl N-benzylcarbamoyl-N′-methyl-hydrazino Acetic Acid

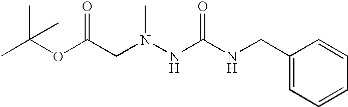

A 3 L, two-necked, round-bottom-flask was fitted with a glass stopper and reflux condenser connected to a calcium tube. A suspension of N-benzylcarbamoyl-N-methylhydrazine (72 g, 0.402 mol) in 900 mL of Tlouene:DMF (800 mL:100 mL=v/v: 8/1) co-solvent was added to the flask and then was added K₂CO₃ (281 g). After heating at 70˜80° C. for 1 hr, a solution of t-Butylbromoacetate (1.1 eq) in Toluene (100 ml) was added slowly and stirred at 70˜80° C. for 5 hrs. The mixture was filtered and extracted with EtOAc (1.0 L×3). The organic solution was washed with Brine (1.0 L×3), evaporated and the residue was slurried with hexane and EtOAc to get a white solid. The solid was filtered and washed with minimum amount of hexane and dried under vacuum for overnight to give the title compound (105 g, 90% as white solid). ¹H NMR (CDCl₃, 300 MHz): δ1.46 (9H, s), δ2.70 (3H, s), δ3.42 (2H, br), δ4.45 (2H, d, J=6 Hz), δ6.22 (1H, br) δ6.41 (1H, t, J=6 Hz), δ7.31 (5H, m)

### (5) Preparation N-benzylcarbamoyl-N′-methyl-hydrazino Acetic Acid

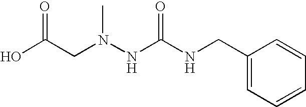

A 3 L, two-necked, round-bottom-flask was fitted with a glass stopper and a calcium chloride tube. A solution of N-benzylcarbamoyl-N′-methyl-hydrazino acetic acid t-butyl ester (105 g, 0.358 mol) in HCl (200 mL, 4 M solution in Dioxane) was added and stirred vigorously under ice-water external bath and then warmed to room temperature. 1,4-dioxane (2.0 L) was added in the reaction solution. After stirring at room temperature for overnight, the solution was concentrated completely by rotary evaporation at 40° C. at aspirator vacuum. The saturated aq. NaHCO₃ solution was added and the aqueous layer was washed with EtOAc (1.0 L×2). Conc. HCl was added dropwise slowly at 0° C. (pH 2-3). The mixture was extracted with EtOAc (1.0 L×2), and the organic layer was dried over sodium sulfate and evaporated. The residue was purified by crystallization with n-hexane and EtOAc to give the title compound and evaporated in vacuo (to obtain 75 g, 89% as white solid). ¹H NMR (CDCl₃): δ2.79 (3H, s) δ3.46˜3.58 (2H, br), δ4.43 (2H, d, J=6 Hz), δ6.53 (1H, t, J=6 Hz), δ7.29 (5H, m), δ7.80 (1H, s), δ12.38 (1H, br)

### Preparation Example 2

### Preparation of N-benzylcarbamoyl-N′-4-fluorobenzyl-hydrazino Acetic Acid

### (1) Preparation of N-benzylcarbamoyl-N′-Boc-hydrazine

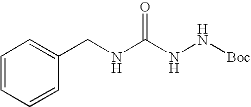

A solution of Boc-Carbazate (200 g, 1.51 mol) in THF (2.0 L) was added and a solution of benzyl isocyanate (1.1 eq.) in THF was added. After 6 hr, The solution was evaporated in vacuo. The residue was slurried with Hexane/EA to get a white solid. The colorless solid that separated was filtered and washed with minimum amount of Hexane and dried under vacuum for overnight to give the title compound (white solid, 380 g, 95%). ¹H NMR (DMSO-d₆, 300 MHz) δ 2.89˜3.08 (2H, m), 4.19 (4H, m), 7.27˜7.40 (8H, m), 7.64˜7.73 (2H, m), 7.87 (2H, d, J=6 Hz)

### (2) Preparation of N-benzylcarbamoyl-hydrazine

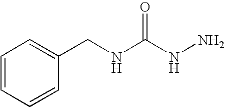

The above compound (380 g, 1.43 mol) was added. A solution of HCl (1 L, 4 M solution in Dioxane) and 1,4-dioxane (3 L) were added slowly with vigorous stirring in an ice water bath. The reaction mixture was stirred at RT over 6 hrs. The solution was concentrated completely by rotary evaporation at 40° C. at aspirator vacuum. The residue was slurried with Hexane/EA to get a white solid. The colorless solid that separated was filtered and washed with minimum amount of Hexane and dried under vacuum for overnight to give the title compound (white solid, 270 g, 94%). ¹H NMR (DMSO-d₆, 300 MHz) δ 2.89˜3.08 (2H, m), 4.19 (4H, m), 7.27˜7.40 (8H, m), 7.64˜7.73 (2H, m), 7.87 (2H, d, J=6 Hz)

### (3) Preparation of t-Butyl N-benzylcarbamoyl-hydrazino Acetic Acid

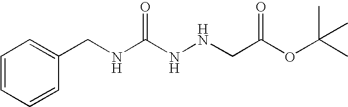

A 10 L, two-necked, round-bottom-flask was fitted with a glass stopper and reflux condenser connected to a calcium tube. A solution of N-Benzylcarbamoyl-hydrazine (270 g, 1.34 mol) in 4 L of Toluene: DMF (3 L: 1 L) co-solvent was added. Diisopropylethylamine (1.1 eq.) was added slowly at 0° C. for 30 min. The reaction mixture was warmed to RT. and K₂CO₃ (3.0 eq.) was added slowly. A solution of t-Butylbromoacetate (1.1 eq) in Toluene was added through a dropping funnel. The reaction mixture was stirred at 70° C.˜80° C. for 6 hrs. The mixture was filtered and extracted with EtOAc (4.0 L). The organic layer was washed with Brine (2 Times), evaporated in vacuo and the residue was slurried with hexane and EtOAc to get a white solid. The solid was filtered and washed with minimum amount of hexane and dried under vacuum for overnight to give the title compound (260 g, 70% as white solid). ¹H NMR (CDCl₃, 300 MHz) δ 1.47 (9H, s), 3.41 (2H, d), 4.13 (1H, br, t), 4.41 (2H, d), 6.31 (1H, br), 6.39 (1H, br), 7.31 (5H, m)

### (4) Preparation of t-Butyl N-benzylcarbamoyl-N′-4-fluorobenzyl-hydrazino Acetic Acid

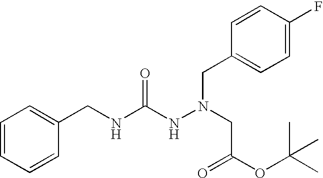

Secondary Hydrazine (74 g, 265 mmol) was dissolved in DMF/Toluene (v/v=1/3) 2 L. A 5 L, two-necked, round-bottom-flask was fitted with a glass stopper and reflux condenser connected to a calcium tube then K₂CO₃ (3.0 eq.) was added in the reaction mixture. The solution was heated to 70° C.˜80° C. for 30 min and 4-fluorobenzyl bromide (1.1 eq) in Toluene was added through a dropping funnel. The reaction mixture was stirred at 70° C.˜80° C. for 6 hrs. The mixture was filtered and extracted with EtOAc (2.0 L). The organic layer was washed with Brine (2 Times), evaporated in vacuo and the residue was slurried with hexane and EtOAc to get a white solid. The solid was filtered and washed with minimum amount of hexane and dried under vacuum for overnight to give the title compound (white solid, 81 g, 79%). ¹H NMR (CDCl₃, 300 MHz) δ 1.47 (9H, s), 3.44 (2H, br), 3.97 (2H, br), 4.30 (2H, br), 6.16 (1H, br), 6.32 (1H, br), 6.96 (2H, m), 7.11 (2H, d), 7.27 (5H, m)

### (5) Preparation of N-benzylcarbamoyl-N′-4-fluorobenzyl-hydrazino Acetic Acid

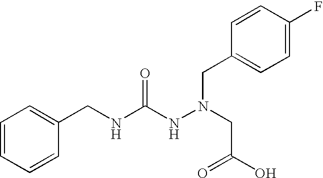

A 3 L round-bottom-flask was fitted with a glass stopper and a calcium tube. N-benzylcarbamoyl-N′-4-fluorobenzyl-hydrazino acetic acid t-butyl ester (81 g, 0.209 mol) was added. A solution of HCl (500 mL, 4 M solution in dioxane) and 1,4-dioxane (1 L) were added slowly with vigorous stirring in an ice water bath. The reaction mixture was stirred at RT over 6 hrs. The solution was concentrated completely by rotary evaporation at 40° C. at aspirator vacuum. The residue was slurried with Hexane/EA to get a white solid. The colorless solid that separated was filtered and washed with minimum amount of Hexane and dried under vacuum for overnight to give the title compound (56 g, 81% as white solid). ¹H NMR (DMSO-d₆, 300 MHz) δ 3.53 (2H, s), 3.87 (2H, s), 4.11 (2H, d), 6.81 (1H, t), 7.01 (4H, m), 7.19 (4H, m), 7.40 (2H, m)

### Preparation Example 3

### Preparation of N-benzylcarbamoyl-N′-4-benzyl-hydrazino Acetic Acid

### (1) Preparation of N-benzylcarbamoyl-N′-Boc-hydrazine

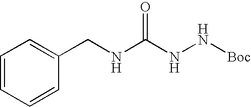

A solution of Boc-Carbazate (200 g, 1.51 mol) in THF (2.0 L) was added and a solution of benzyl isocyanate (1.1 eq.) in THF was added. After 6 hr, The solution was evaporated in vacuo. The residue was slurried with Hexane/EA to get a white solid. The colorless solid that separated was filtered and washed with minimum amount of Hexane and dried under vacuum for overnight to give the title compound (white solid, 380 g, 95%). ¹H NMR (DMSO-d₆, 300 MHz) δ 2.89˜3.08 (2H, m), 4.19 (4H, m), 7.27˜7.40 (8H, m), 7.64˜7.73 (2H, m), 7.87 (2H, d, J=6 Hz)

### (2) Preparation of N-benzylcarbamoyl-hydrazine

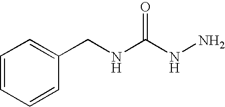

The above compound (380 g, 1.43 mol) was added. A solution of HCl (1 L, 4 M solution in dioxane) and 1,4-dioxane (3 L) were added slowly with vigorous stirring in an ice water bath. The reaction mixture was stirred at RT over 6 hrs. The solution was concentrated completely by rotary evaporation at 40° C. at aspirator vacuum. The residue was slurried with Hexane/EA to get a white solid. The colorless solid that separated was filtered and washed with minimum amount of Hexane and dried under vacuum for overnight to give the title compound (white solid, 270 g, 94%) ¹H NMR (DMSO-d₆, 300 MHz) δ 2.89˜3.08 (2H, m), 4.19 (4H, m), 7.27˜7.40 (8H, m), 7.64˜7.73 (2H, m), 7.87 (2H, d, J=6 Hz)

### (3) Preparation of t-Butyl N-benzylcarbamoyl-hydrazino Acetic Acid

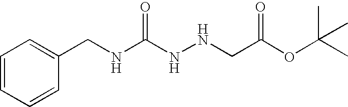

A 10 L, two-necked, round-bottom-flask was fitted with a glass stopper and reflux condenser connected to a calcium tube. A solution of N-benzylcarbamoyl-hydrazine (270 g, 1.34 mol) in 4 L of Toluene:DMF (3 L:1 L) co-solvent was added. Diisopropylethylamine (1.1 eq.) was added slowly at 0° C. for 30 min. The reaction mixture was warmed to RT. and K₂CO₃ (3.0 eq.) added slowly. A solution of t-Butylbromoacetate (1.1 eq) in Toluene was added through a dropping funnel. The reaction mixture was stirred at 70° C.˜80° C. for 6 hrs. The mixture was filtered and extracted with EtOAc (4.0 L). The organic layer was washed with Brine (2 Times), evaporated in vacuo and the residue was slurried with hexane and EtOAc to get a white solid. The solid was filtered and washed with minimum amount of hexane and dried under vacuum for overnight to give the title compound (260 g, 70% as white solid). ¹H NMR (CDCl₃, 300 MHz) δ 1.47 (9H, s), 3.41 (2H, d), 4.13 (1H, br, t), 4.41 (2H, d), 6.31 (1H, br), 6.39 (1H, br), 7.31 (5H, m)

### (4) Preparation of t-Butyl N-benzylcarbamoyl-N′-4-benzyl-hydrazino Acetic Acid

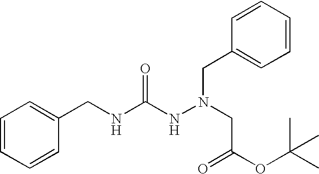

Secondary Hydrazine (74 g, 265 mmol) was dissolved in 2 L of DMF:Toluene (1.5 L: 0.5 L). A 5 L, two-necked, round-bottom-flask was fitted with a glass stopper and reflux condenser connected to a calcium tube then K₂CO₃ (3.0 eq.) was added in the reaction mixture. The solution was heated to 70° C.˜80° C. for 30 min and benzylbromide (1.1 eq) in Toluene was added through a dropping funnel. The reaction mixture was stirred at 70° C.˜80° C. for 6 hrs. The mixture was filtered and extracted with EtOAc (2.0 L). The organic layer was washed with Brine (2 Times) and evaporated in vacuo and the residue was slurried with hexane and EtOAc to get a white solid. The solid was filtered and washed with minimum amount of hexane and dried under vacuum for overnight to give the title compound (white solid, 81 g, 79%). ¹H NMR (CDCl₃, 300 MHz) δ 1.47 (9H, s), 3.44 (2H, br), 3.97 (2H, br), 4.30 (2H, br), 6.16 (1H, br), 6.32 (1H, br), 7.27 (10H, m)

### (5) Preparation of N-benzylcarbamoyl-N′-4-benzyl-hydrazino Acetic Acid

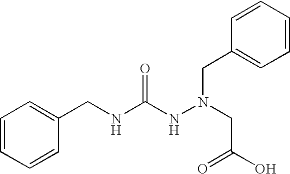

A 3 L round-bottom-flask was fitted with a glass stopper and a calcium tube. N-Benzylcarbamoyl-N′-4-benzyl-hydrazino acetic acid t-butyl ester (81 g, 0.209 mol) was added. A solution of HCl (500 mL, 4 M solution in Dioxane) and 1,4-dioxane (1 L) were added slowly with vigorous stirring in an ice water bath. The reaction mixture was stirred at RT over 6 hrs. The solution was concentrated completely by rotary evaporation at 40° C. at aspirator vacuum. The residue was slurried with Hexane/EA to get a white solid. The colorless solid that separated was filtered and washed with minimum amount of Hexane and dried under vacuum for overnight to give the title compound (56 g, 81% as white solid). ¹H NMR (DMSO-d₆, 300 MHz) δ 3.53 (2H, s), 3.87 (2H, s), 4.11 (2H, d), 6.81 (1H, t), 7.27 (10H, m).

### Preparation Example 4

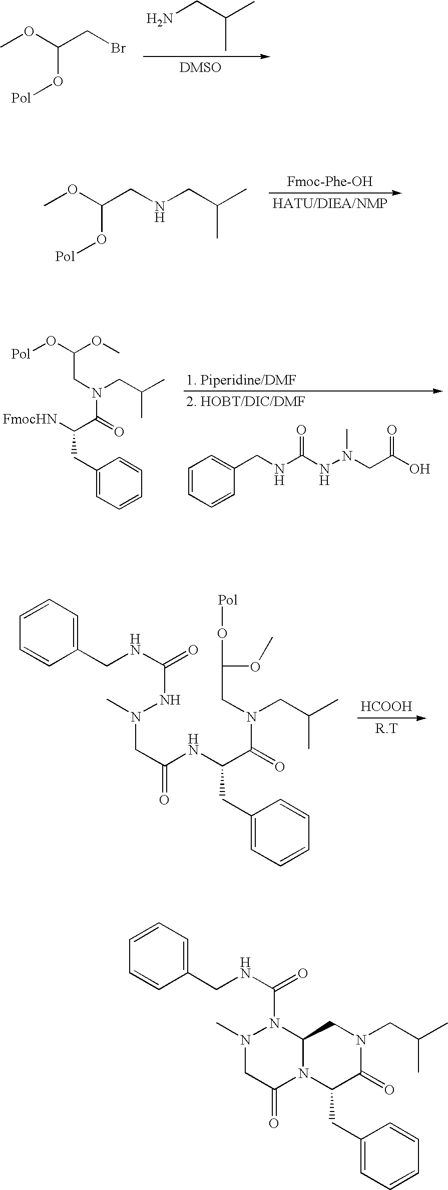

### (1) Preparation of 8-(3-chloro-2-dimethylamino-benzyl)-6-(4-hydroxy-benzyl)-2-methyl-4,7-dioxo-hexa hydro-pyrazino[2,1-c][1,2,4]triazine-1-carboxylic Acid Benzyl Amide

Bromoacetal resin (60 mg, 0.98 mmol/g) and a solution of (2-dimethylamino-6-chloro-phenyl)-methyl-amine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM, to provide a first component piece.

A solution of Fmoc-Tyrosine (OtBu)-OH (4 equiv., commercially available, second component piece), HATU (PerSeptive Biosystems, 4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of N-benzylcarbamoyl-N′-methyl-hydrazino acetic acid (4 equiv., third component piece), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, DCM, and then MeOH. The resin was dried in vacuo at room temperature.

The resin was treated with formic acid (2.5 ml) for 18 hours at room temperature. After the resin was removed by filtration, the filtrate was condensed under reduced pressure to give the title compound as an oil. ¹H-NMR (300 MHz, CDCl₃) δ ppm; 2.45 (s, 3H), 2.65 (s, 6H), 3.12 (m, 2H), 3.52 (m, 4H), 4.12 (dd, 1H), 4.24 (dd, 1H), 4.45 (d, 1H), 4.75 (d, 1H), 5.20 (t, 1H), 5.56 (dd, 1H), 6.40 (m, 3H), 6.66 (d, 2H), 7.11 (d, 2H), 7.39 (m, 5H)

### Preparation Example 5

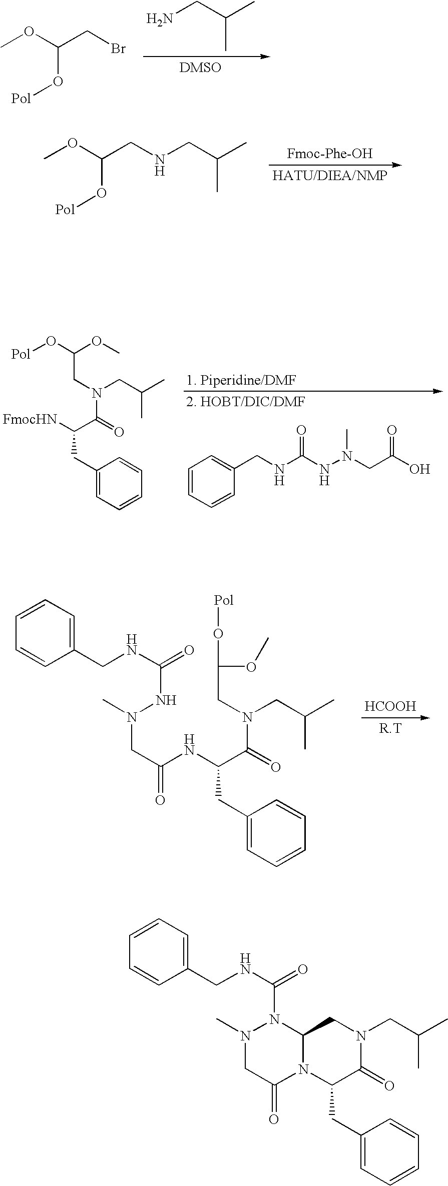

### (1) Preparation of 8-isobutyl-2-methyl-4,7-dioxo-6-phenyl-hexahydro-pyrazino 2,1-c][1,2,4]triazine-1-carboxylic Acid Benzyl Amide

Bromoacetal resin (60 mg, 0.98 mmol/g) and a solution of (2-aminomethyl-6-chloro-phenyl)-dimethyl-amine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM, to provide a first component piece.

A solution of Fmoc-phenylalanine-OH (4 equiv., commercially available, second component piece), HATU (PerSeptive Biosystems, 4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of N-benzylcarbamoyl-N′-methyl-hydrazino acetic acid (4 equiv., third component piece), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, DCM, and then MeOH. The resin was dried in vacuo at room temperature. The resin was treated with formic acid (2.5 ml) for 18 hours at room temperature. After the resin was removed by filtration, the filtrate was condensed under reduced pressure to give the title compound as an oil. ¹H-NMR (300 MHz, CDCl₃) δ ppm; 1.12 (s, 6H), 2.45 (s, 3H), 2.47 (m, 1H), 3.15 (m, 4H), 3.47 (m, 4H), 4.10 (d, 1H), 4.23 (d, 1H), 5.15 (t, 1H), 6.03 (dd, 1H), 7.15 (m, 5H), 7.23 (m, 5H)

### Preparation Example 6

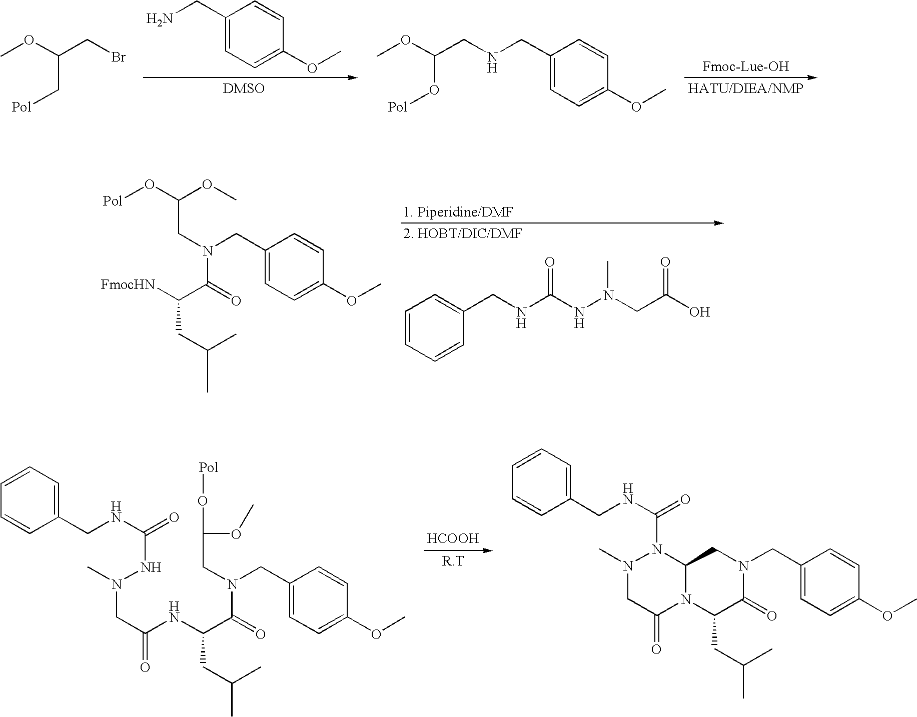

### (1) Preparation of 6-isobutyl-8-(4-methoxy-benzyl)-2-methyl-4,7-dioxo-hexahydro-pyrazino[2,1-c][1,2,4]triazine-1-carboxylic Acid Benzyl Amide

The same procedure as that described in preparation example 5 was performed except that Fmoc-leucine-OH was used instead of Fmoc-phenylalanine-OH, to give the title compound as an oil. ¹H-NMR (300 MHz, CDCl₃) δ ppm; 1.10 (s, 6H), 1.80 (m, 3H), 2.45 (s, 3H), 3.40 (m, 2H), 3.58 (m, 2H), 3.70 (s, 3H), 4.45 (dd, 1H), 4.48 (dd, 1H), 4.50 (s, 2H), 4.57 (t, 1H), 6.28 (dd, 1H), 6.70 (d, 2H), 6.98 (d, 2H), 7.18 (m, 5H)

### Preparation Example 7

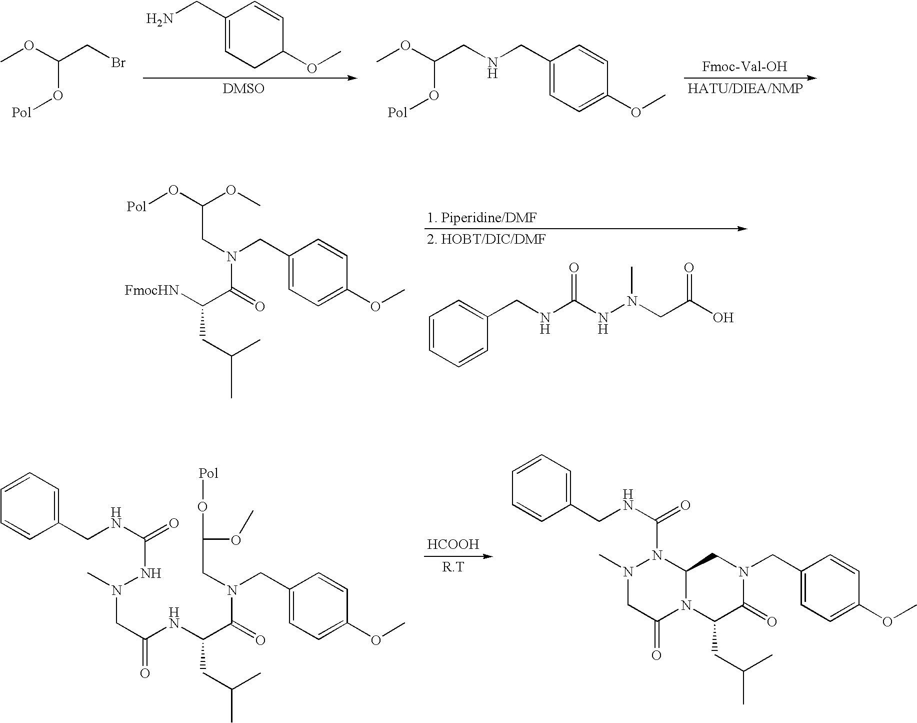

### (1) Preparation of (6S,9aS)-N-benzyl-6-isobutyl-8-(4-methoxybenzyl)-2-methyl-4,7-dioxo-hexahydro-2H-pyrazino[2,1-c][1,2,4]triazine-1(6H)-carboxamide

Bromoacetal resin (60 mg, 0.98 mmol/g) and a solution of 4-methoxy-benzyl amine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM, to provide a first component piece.

A solution of Fmoc-Val-OH (4 equiv), HATU (PerSeptive Biosystems, 4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of 2-(2-(benzylcarbamoyl)-1-methylhydrazinyl)acetic acid (4 equiv.), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, DCM, and then MeOH. The resin was dried in vacuo at room temperature.

The resin was treated with formic acid (2.5 ml) for 18 hours at room temperature. After the resin was removed by filtration, the filtrate was condensed under reduced pressure to give the title compound as an oil. ¹H-NMR (400 MHz, CDCl₃) δ ppm; 1.51 (d, 6H), 1.91 (m, 1H), 2.49 (s, 3H), 3.45 (d, 2H), 3.69 (m, 2H), 3.72 (s, 3H), 3.82 (m, 2H), 4.40 (s, 2H), 4.48 (s, 2H), 4.53 (t, 1H), 5.55 (t, 1H), 6.61 (d, 2H), 6.95 (d, 2H), 7.24-7.38 (m, 5H).

### Preparation Example 8

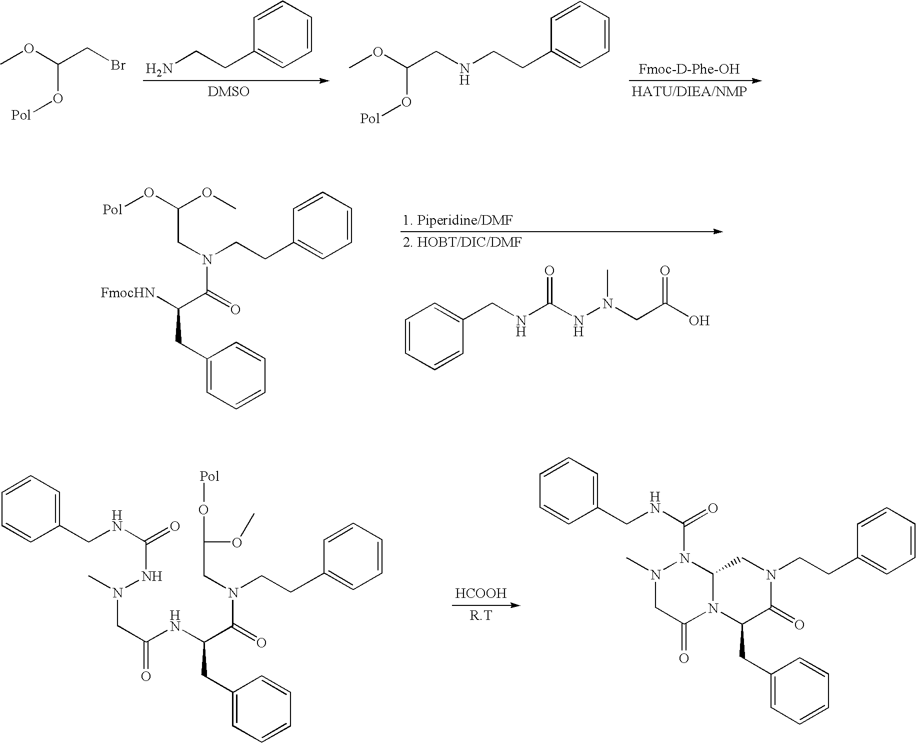

### (1) Preparation of (6R,9aR)-N,6-dibenzyl-2-methyl-4,7-dioxo-8-phenethyl-hexahydro-2H-pyrazino[2,1-c][1,2,4]triazine-1(6H)-carboxamide

Bromoacetal resin (60 mg, 0.98 mmol/g) and a solution of 2-phenylethanamine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM, to provide a first component piece.

A solution of Fmoc-D-Phe-OH (4 equiv., commercially available, second component piece), HATU (PerSeptive Biosystems, 4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of 2-(2-(benzylcarbamoyl)-1-methylhydrazinyl)acetic acid (4 equiv.), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, DCM, and then MeOH. The resin was dried in vacuo at room temperature.

The resin was treated with formic acid (2.5 ml) for 18 hours at room temperature. After the resin was removed by filtration, the filtrate was condensed under reduced pressure to give the title compound as an oil. ¹H-NMR (400 MHz, CDCl₃) δ ppm; 2.47 (s, 3H), 2.81 (t, 2H), 2.90 (dd, 1H), 3.15 (dd, 1H), 3.47-3.58 (m, 5H), 3.81 (m, 1H), 4.47 (s, 2H), 4.97 (t, 1H), 5.80 (t, 1H), 7.15 (m, 2H), 7.21-7.38 (m, 13H).

### Preparation Example 9

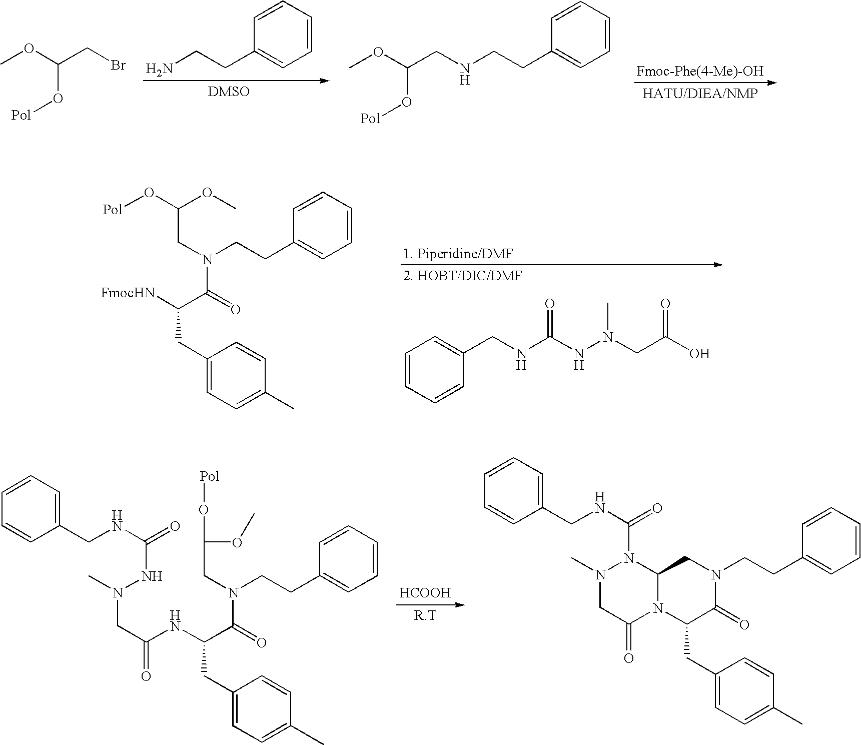

### (1) Preparation of (6S,9aS)-N-benzyl-2-methyl-6-(4-methylbenzyl)-4,7-dioxo-8-phenethyl-hexahydro-2H-pyrazino[2,1-c][1,2,4]triazine-1(6H)-carboxamide

The same procedure as that described in preparation example 7 was performed except that Fmoc-Phe(4-Me)-OH was used instead of Fmoc-D-Phe-OH, to give the title compound as an oil. ¹H-NMR (400 MHz, CDCl₃) δ ppm; 2.19 (s, 3H), 2.51 (s, 3H), 2.92 (t, 2H), 2.93 (dd, 1H), 3.20 (dd, 1H), 3.42-3.60 (m, 5H), 3.85 (m, 1H), 4.45 (s, 2H), 4.92 (t, 1H), 5.80 (t, 1H), 7.00 (d, 4H), 7.25-7.35 (m, 10H).

### Preparation Example 10

### Preparation of 2-(4-Fluoro-benzyl)-4,7-dioxo-6-phenethyl-8-(3-trifluoro-methylbenzyl)-hexahydro-pyrazino[2,1-c][1,2,4]triazine-1-carboxylic Acid Benzylamide

Bromoacetal resin (60 mg, 0.98 mmol/g) and a solution of 3-Trifluoro-methyl benzylamine naphthyl amine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM.

A solution of Fmoc-homophenylalanine (4 equiv.), HATU [PerSeptive Biosystems] (4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of N^{ε}-Fmoc-N^{α}-fluorobenzyl-hyrazinoglycine (4 equiv.), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, and then DCM. To the resin was added 20% piperidine in DMF (10 ml for 1 g of the resin). After the reaction mixture was shaken for 8 min at room temperature, the resin was collected by filtration and washed with DMF, DCM, and then DMF.

The resin was treated with a mixture of benzyl isocyanate (4 equiv.) and DIEA (4 equiv.) in DCM for 4 hours at room temperature. Then, the resin was collected by filtration and washed with DMF, DCM, and then MeOH. After the resin was dried in vacuo at room temperature, the resin was treated with formic acid (2.5 ml) for 18 hours at room temperature. The resin was removed by filtration, and the filtrate was condensed under reduced pressure to give the title compound as an oil.

¹H-NMR (400 MHz, CDCl₃) δ ppm; 3.15-3.53 (m, 6H), 4.31-4.42 (m, 4H), 4.47-4.85 (m, 2H), 5.22 (t, 1H), 5.47 (m, 2H), 6.81 (d, 2H), 6.85 (d, 2H), 6.91 (m, 4H), 7.15-8.24 (m, 14H);

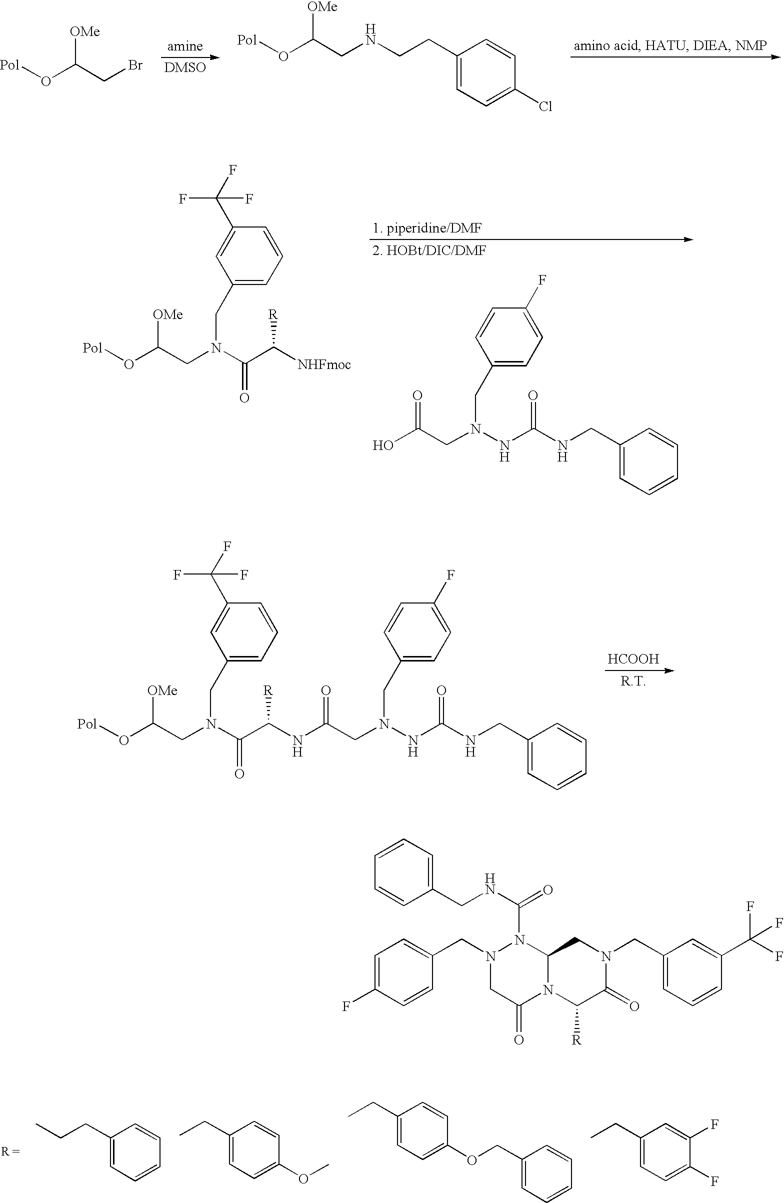

### Preparation Example 11

### Preparation of 2-(4-Fluoro-benzyl)-6-(4-methoxy-benzyl)-4,7-dioxo-8-(3-trifluoromethyl-benzyl)-hexahydro-pyrazino[2,1-c][1,2,4]triazine-1-carboxylic Acid Benzylamide

Bromoacetal resin (60 mg, 0.98 mmol/g) and a solution of 3-Trifluoro-methyl benzylamine naphthyl amine in DMSO (2.5 ml, 2 M) were placed in a vial with screw cap. The reaction mixture was shaken at 60° C. using rotating oven [Robbins Scientific] for 12 hours. The resin was collected by filtration, and washed with DMF, then DCM.

A solution of Fmoc-Tyr(O-Me) (4 equiv.), HATU [PerSeptive Biosystems] (4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of N^{β}-Fmoc-N^{α}-4-fluorobenzyl-hyrazinoglycine (4 equiv.), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, and then DCM. To the resin was added 20% piperidine in DMF (10 ml for 1 g of the resin). After the reaction mixture was shaken for 8 min at room temperature, the resin was collected by filtration and washed with DMF, DCM, and then DMF.

The resin was treated with a mixture of benzyl isocyanate (4 equiv.) and DIEA (4 equiv.) in DCM for 4 hours at room temperature. Then, the resin was collected by filtration and washed with DMF, DCM, and then MeOH. After the resin was dried in vacuo at room temperature, the resin was treated with formic acid (2.5 ml) for 18 hours at room temperature. The resin was removed by filtration, and the filtrate was condensed under reduced pressure to give the title compound as an oil.

¹H-NMR (400 MHz, CDCl₃) δ ppm; 3.24-3.52 (m, 4H), 3.98 (s, 3H) 4.19-4.25 (m, 4H), 4.47-4.85 (m, 2H), 5.33 (t, 1H), 5.35 (m, 2H), 6.78 (d, 2H), 6.88 (d, 2H), 6.91 (m, 4H), 7.05-8.11 (m, 9H);

### Preparation Example 12

### Preparation of 6-(4-benzyloxy-benzyl)-2-(4-fluoro-benzyl)-4,7-dioxo-8-(3-trifluoromethyl-benzyl)-hexahydro-pyrazino[2,1-c][1,2,4]triazine-1-carboxylic Acid Benzylamide

The same procedure as that described in preparation example 11 was performed except that Fmoc-Tyr(O-Bn) was used instead of Fmoc-Tyr(O-Me), to give the title compound as an oil.

¹H-NMR (400 MHz, CDCl₃) δ ppm; 3.10-3.55 (m, 4H), 4.31-4.45 (m, 6H), 4.47-4.85 (m, 2H), 5.20 (t, 1H), 5.44 (m, 2H), 6.66 (d, 2H), 6.67 (d, 2H), 7.25-8.03 (m, 18H);

### Preparation Example 13

### Preparation of 6-(3,4-difluoro-benzyl)-2-(4-fluoro-benzyl)-4,7-dioxo-8-(3-trifluoromethyl-benzyl)-hexahydro-pyrazino[2,1-c][1,2,4]triazine-1-carboxylic Acid Benzylamide

The same procedure as that described in preparation example 11 was performed except that Fmoc-3,4-difluoro Phe was used instead of Fmoc-Tyr(O-Me), to give the title compound as an oil.

¹H-NMR (400 MHz, CDCl₃) δ ppm; 3.15-3.53 (m, 4H), 4.31-4.42 (m, 6H), 4.47-4.85 (m, 2H), 5.22 (t, 1H), 5.47 (m, 2H), 6.68 (d, 2H), 6.99 (d, 2H), 7.21-8.21 (m, 12H);

### Preparation Example 14

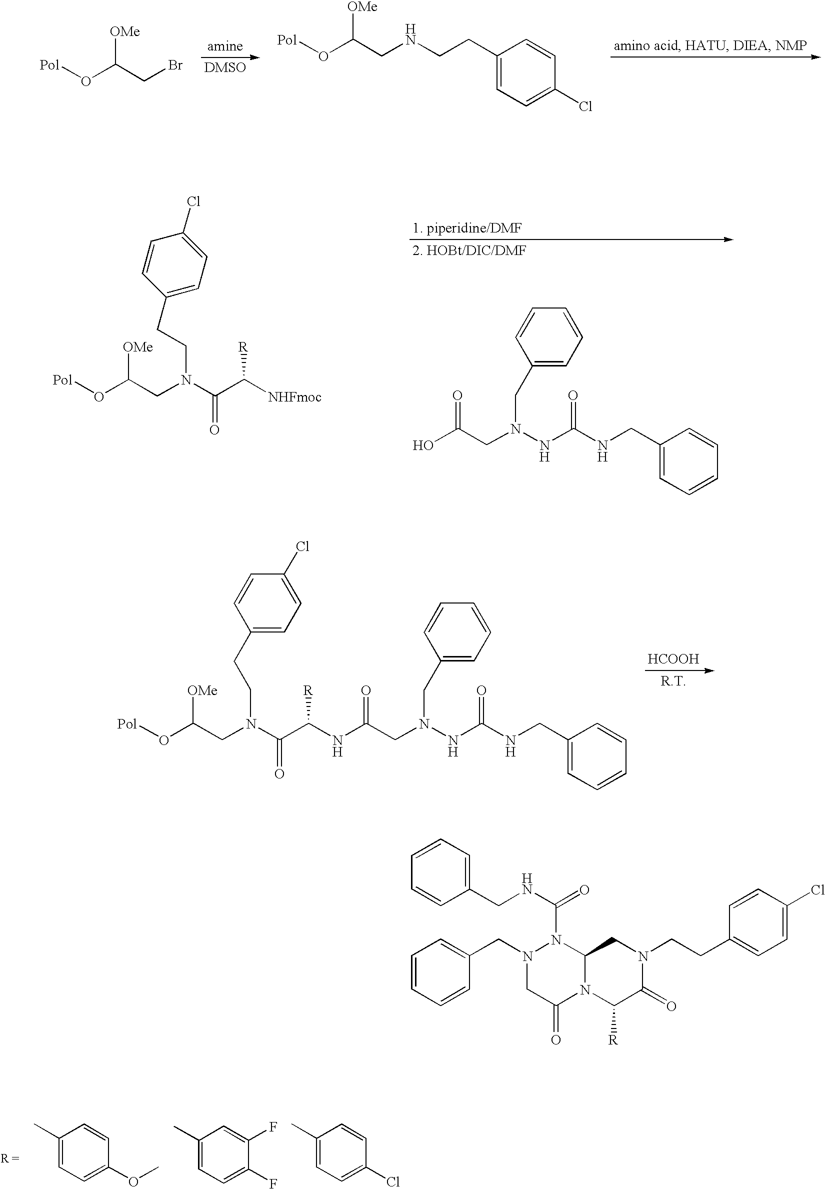

### Preparation of 2-benzyl-8-[2-(4-chloro-phenyl)-ethyl]-6-(4-methoxy-benzyl)-4,7-dioxo-hexahydro-pyrazino[2,1-c][1,2,4]triazine-1-carboxylic Acid Benzylamide

Bromoacetal resin (60 mg, 0.98 mmol/g) and a solution of 2-(4-chloro-phenyl)-ethylamine (2.5 ml, 2 M) were placed in a vial with screw cap. The reaction mixture was shaken at 60° C. using rotating oven [Robbins Scientific] for 12 hours. The resin was collected by filtration, and washed with DMF, then DCM, to provide a first component piece.

A solution of 2-(9H-Fluoren-9-ylmethoxycarbonylamino)-3-(4-methoxy-phenyl)-propionic acid (4 equiv., commercially available, second component piece), HATU (PerSeptive Biosystems, 4 equiv.), and DIEA (4 equiv.) in NMP (Advanced ChemTech) was added to the resin. After the reaction mixture was shaken for 4 hours at room temperature, the resin was collected by filtration and washed with DMF, DCM, and then DMF.

To the resin was added 20% piperidine in DMF. After the reaction mixture was shaken for 8 min at room temperature, the resin was collected by filtration and washed with DMF, DCM, and then DMF.

A solution of 2-(1-benzylcarbamoyl)hydrazinyl)acetic acid (4 equiv.), HOBT [Advanced ChemTech] (4 equiv.), and DIC (4 equiv.) in DMF was added to the resin prepared above. After the reaction mixture was shaken for 3 hours at room temperature, the resin was collected by filtration and washed with DMF, DCM, and then MeOH. The resin was dried in vacuo at room temperature.

The resin was treated with formic acid (2.5 ml) for 18 hours at room temperature. After the resin was removed by filtration, the filtrate was condensed under reduced pressure to give the title compound as an oil. ¹H-NMR (400 MHz, CDCl₃) δ ppm; 3.22-3.57 (m, 10H), 3.82 (s, 3H), 4.36 (d, 2H), 4.50 (d, 1H), 4.90 (d, 1H), 5.36-5.45 (m, 2H), 6.62-6.73 (m, 4H), 6.98-7.05 (m, 4H), 7.10-7.48 (m, 10H).

### Preparation Example 15

### Preparation of 2-benzyl-8-[2-(4-chloro-phenyl)-ethyl]-6-(3,4-difluoro-benzyl)-4,7-dioxo-hexahydro-pyrazino[2,1-c][1,2,4]triazine-1-carboxylic Acid Benzylamide

The same procedure as that described in preparation example 14 was performed except that 3-(3,4-difluoro-phenyl)-2-(9H-fluoren-9-ylmethoxycarbonylamino)-propionic acid was used instead of 2-(9H-Fluoren-9-ylmethoxycarbonylamino)-3-(4-methoxy-phenyl)-propionic acid, to give the title compound as an oil. ¹H-NMR (400 MHz, CDCl₃) δ ppm; 3.25-3.68 (m, 9H), 4.36 (d, 2H), 4.52 (d, 1H), 4.96 (d, 1H), 5.36-5.45 (m, 2H), 6.50-6.76 (m, 2H), 6.92-7.08 (m, 5H), 7.10-7.48 (m, 10H).

### Preparation Example 16

### Preparation of 2-benzyl-6-(4-chloro-benzyl)-8-[2-(4-chloro-phenyl)-ethyl]-4,7-dioxo-hexahydro-pyrazino[2,1-c][1,2,4]triazine-1-carboxylic Acid Benzylamide

The same procedure in preparation example 14 was performed except that 3-(4-chloro-phenyl)-2-(9H-fluoren-9-ylmethoxycarbonylamino)-propionic acid was used instead of 2-(9H-fluoren-9-ylmethoxycarbonylamino)-3-(4-methoxy-phenyl)-propionic acid, to give the product as an oil. ¹H-NMR (400 MHz, CDCl3) δ ppm; 3.18-3.50 (m, 9H), 4.41 (d, 2H), 4.50 (d, 1H), 4.90 (d, 1H), 5.36-5.45 (m, 2H), 6.60-6.78 (m, 4H), 6.98-7.05 (m, 4H), 7.15-7.58 (m, 10H).

### Preparation Example 17

### Preparation of 2-methyl-5-(p-hydroxyphenylmethyl)-7-naphthylmethyl-3,6-dioxo-hexahydro-[1,2,4]triazolo[4,5-a]pyrazine-1-carboxylic Acid Benzylamide

Bromoacetal resin (30 mg, 0.98 mmol/g) and a solution of naphthylmethyl amine in DMSO (1.5 ml, 2 M) were placed in a vial with screw cap. The reaction mixture was shaken at 60° C. using rotating oven [Robbins Scientific] for 12 hours. The resin was collected by filtration, and washed with DMF, then DCM to provide a first component piece.

A solution of Fmoc-Tyr(OBut)-OH (3 equiv.), HATU (PerSeptive Biosystems, 3 equiv.), and DIEA (3 equiv.) in NMP (Advanced ChemTech) was added to the resin. After the reaction mixture was shaken for 4 hours at room temperature, the resin was collected by filtration and washed with DMF, DCM, and then DMF, to thereby add the second component piece to the first component piece.

To the resin was added 20% piperidine in DMF. After the reaction mixture was shaken for 8 min at room temperature, the resin was collected by filtration and washed with DMF, DCM, and then DMF.

A solution of N′-Fmoc-N-methyl-hydrazinocarbonyl chloride (5 equiv.), DIEA (5 equiv.) in DCM was added to the resin prepared above. After the reaction mixture was shaken for 4 hours at room temperature, the resin was collected by filtration and washed with DMF, DCM, and DMF.

To the resin was added 20% piperidine in DMF (10 ml for 1 g of the resin). After the reaction mixture was shaken for 8 min at room temperature, the resin was collected by filtration and washed with DMF, DCM, and then DMF.

The resin was treated with a mixture of benzyl isocyanate (4 equiv.) and DIEA (4 equiv.) in DCM for 4 hours at room temperature. Then, the resin was collected by filtration and washed with DMF, DCM, and then MeOH. The resin was dried in vacuo at room temperature.

The resin was treated with formic acid for 14 hours at room temperature. After the resin was removed by filtration, the filtrate was condensed under reduced pressure to give the product as an oil.

¹H-NMR (400 MHz, CDCl₃) δ ppm; 2.80-2.98 (m, 5H), 3.21-3.37 (m, 2H), 4.22-4.52 (m, 2H), 4.59 (t, 1H), 4.71 (d, 1H), 5.02 (dd, 1H), 5.35 (d, 1H), 5.51 (d, 1H), 6.66 (t, 2H), 6.94 (dd, 2H), 7.21-8.21 (m, 12H).

### EXAMPLES

### Example 1

### TopFlash Reporter Gene Assay in SW480 Cells

The following test compounds (Compounds A and B) were used in this example.

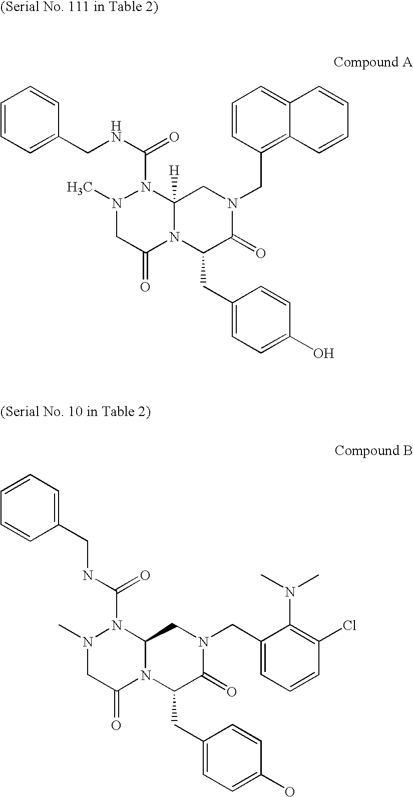

a. Reporter Gene Assay

SW480 cells were transfected using Superfect™ transfection reagent (Qiagen, 301307). Cells were trypsinized briefly 1 day before transfection and plated on 6 well plate (5×10⁵ cells/well) so that they were 50-80% confluent on the day of transfection.

Four microgram (TOPFlash) and one microgram (pRL-null) of DNAs were diluted in 150 ml of serum-free medium, and 30 μl of Superfect™ transfection reagent was added. The DNA-Superfect mixture was incubated at room temperature for 15 min, and then, 1 ml of 10% FBS DMEM was added to this complex for an additional 3 hours of incubation. While complexes were forming, cells were washed with PBS twice without antibiotics.

The DNA-Superfect™ transfection reagent complexes were applied to the cells before incubating at 37° C. at 5% CO₂ for 3 hours. After incubation, recovery medium with 10% FBS was added to bring the final volume to 1.18 ml. After 3 hours incubation, the cells were harvested and reseeded to 96 well plate (3×10⁴ cells/well). After overnight incubation at 37° C. at 5% CO₂, the cells were treated with Compound A or Compound B for 24 hours. Finally, the activity was checked by means of luciferase assay (Promega, E1960).

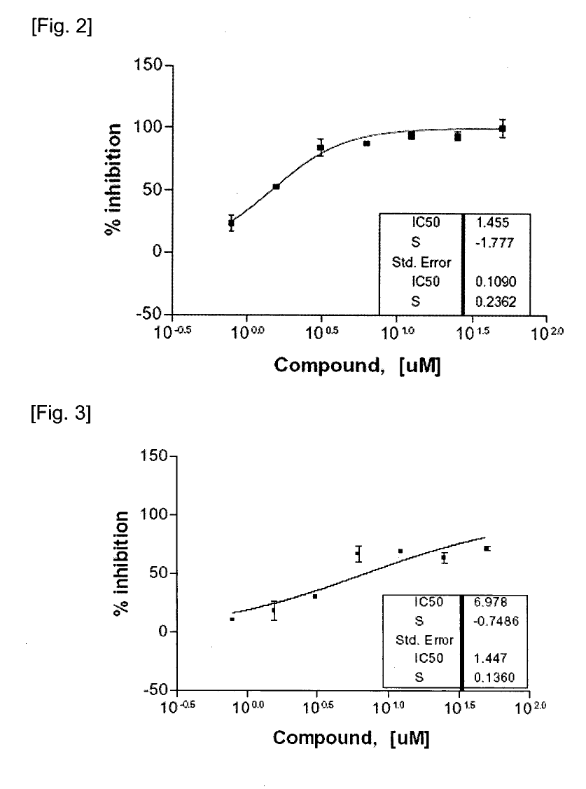

FIGS. 2 and 3 illustrates the results of the measurement of IC₅₀ of Compound A (FIG. 2) and Compound B (FIG. 3) for SW480 cells.

### Example 2

### Modulating Activity of Osteogenesis in Human Mesenchymal Stem Cells

The following compounds (Compound B, Compound C and Compound D) were used in this example:

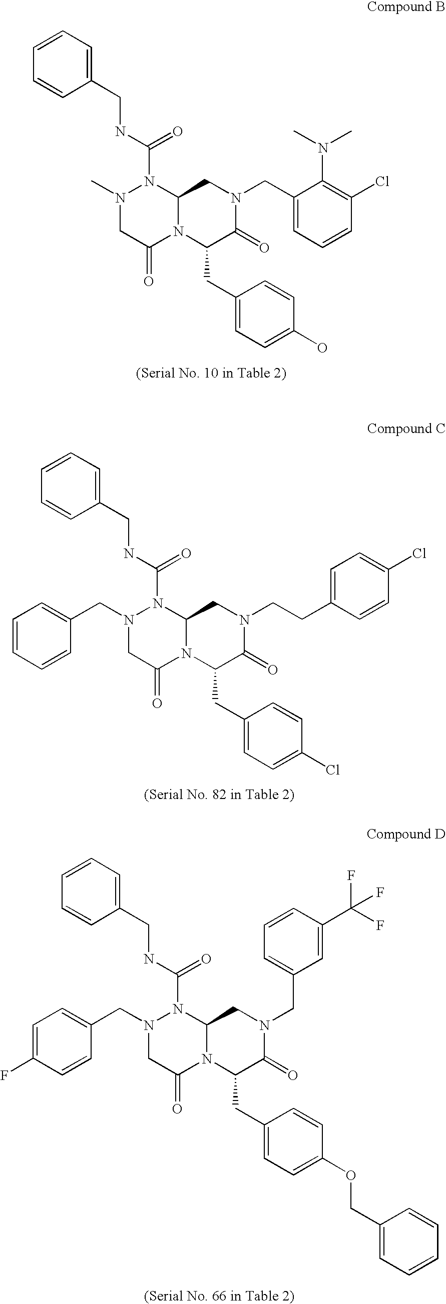

Methods

Cell culture: Human bone marrow derived-mesenchymal stem cells (hBMMSC) were isolated from normal subjects.

Osteogenesis modulating activity assay by test compound alone: hBMMSC were cultivated in the culture medium and cells were seeded into 96-well culture plates and test compounds were dissolved in DMSO and diluted with culture media (final 0.01% DMSO) and added into the media (final concentration of test compound was 20 μM) and cultured in the CO₂ incubator (37° C., 5% CO₂). Culture medium was changed every two days with fresh one and compound was treated again whenever medium was changed. Six to seven days after incubation, Alkaline phosphatase activity of cell lysates was measured by colorimetry. Fold change of ALP activity by each compound compared with DMSO control was summarized in Table 2 (Osteogenesis). For the determination of mineralization within the cells, Von Kossa staining or Alizarin Red S assay was performed 10-14 days after incubation.

Osteogenesis modulating activity of test compounds with induction media: hBMMSC were culture with culture media containing osteogenic induction cocktails (OIC, 0.1 μM dexamethasone, 50 μM ascorbate-2-phosphate, 10 mM b-glycerophosphate). Four to five days after incubation, Alkaline phosphatase (ALP) activity of cell lysates were measured by colorimetry. For the determination of mineralization within the cells, Von Kossa staining or Alizarin Red S assay was performed 10-14 days after incubation.

Cell proliferation assay: To investigate the cytotoxicity of the test compounds for hBMMSC, hBMMSC were exposed to the test compounds for 6-7 days at the same condition above (method b and c). Cell proliferation was assessed using CellTiter 96 Aqueous One Solution (Promega, #G3581). Absorbance at 490 nm of each well was determined with a microplate reader (Molecular Device) and % growth inhibition was calculated compared with control.

RT-PCR. To analyze the mRNA levels for Alkaline phosphatase (ALP), total RNA was isolated using Trizol (Invitrogen-GIBCO-BRL, Baltimore, Md.) from hBMMSC with or without compound treatment for 4 days. 2 μg RNA was reverse transcribed in a total volume of 20 μl with random hexamer (50 ng), and using the Superscript II reverse transcription system (Invitrogen-GIBCO-BRL), according to manufacturer's guidelines. PCR was carried out in a 50 μl volume containing 5 μl cDNA, 100 pmol primers, 100 μM dNTPs, 1×Taq buffer and 1.5 mM MgCl₂. Reaction mixtures were heated to 80° C. for 10 min, after which Taq was added. cDNAs were amplified for 25 (EphB2 receptor) or 15 (GAPDH) cycles. One round of amplification consisted of 1 min at 94° C., 2 min at 60° C., and 2 min at 72° C., with a final extension time of 10 min at 72° C. The PCR products were resolved and visualized by electrophoresis in a 2% gel, stained with ethidium bromide. ALP PCR primers used were, 5′-ATCGGGACTGGTACTCGGATAA-3′ and 5′-ATCAGTTCTGTTCTTCGGGTAC-3′. Primer pairs for GAPDH were 5′-GGTGCTGAGTATGTCGTGGA-3′ and 5′-ACAGTGTTCTGGGTGGCAGT-3′. A housekeeping gene, GADPH was used as a control.

Results

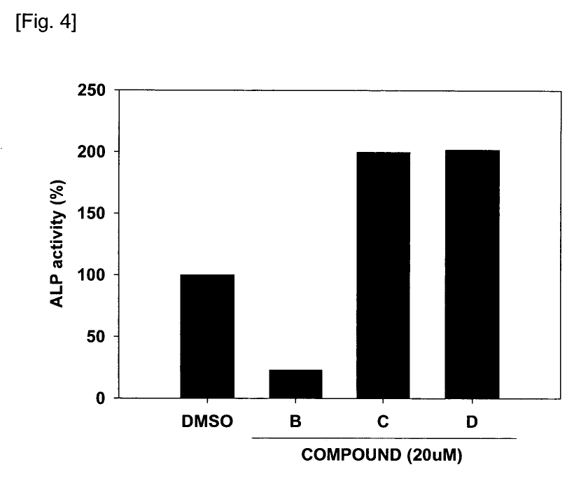

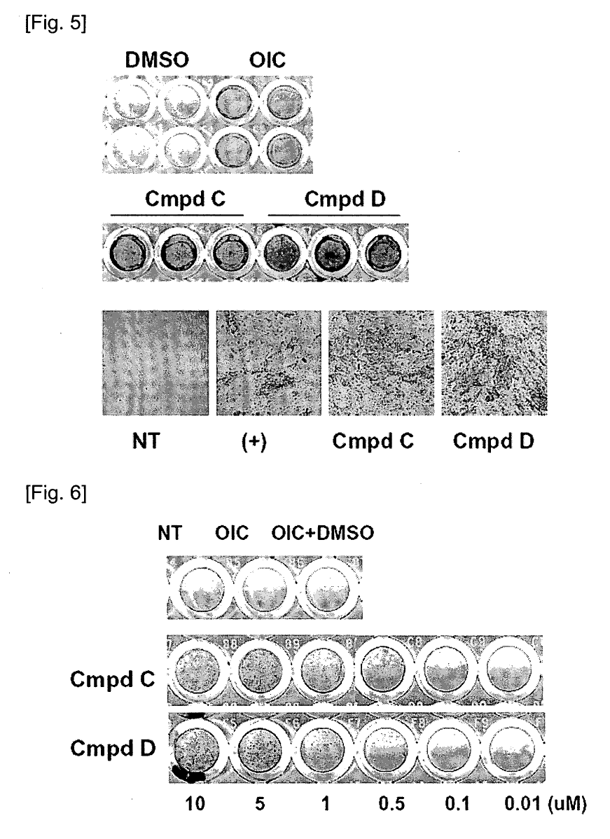

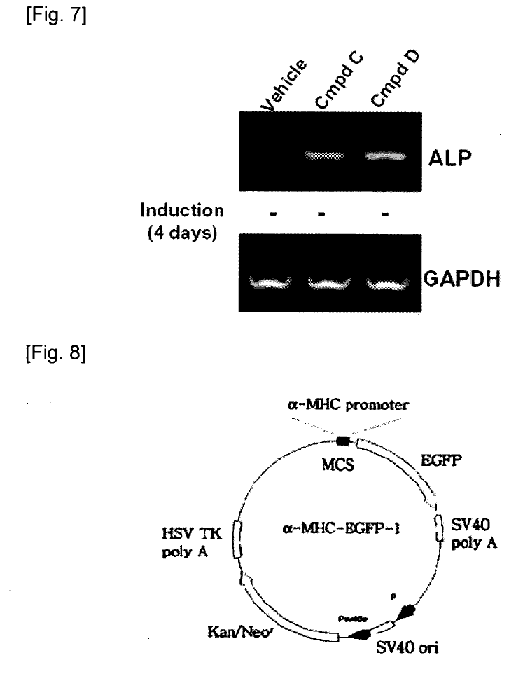

Compound B at 20 μM reduced ALP level (77%) when it was treated alone to hBMMSC (FIG. 4) and cell proliferation was also inhibited (51%). Compound B at 20 μM, when co-treated with osteogenic induction cocktails, reduced ALP level (80%) compared with vehicle control group. On the contrary, Compound C or D at 20 μM significantly enhanced ALP level (200% and 202% respectively) (FIG. 4) and did not significantly affect the cell proliferation (127% and 83% respectively) when it was treated alone. Compound C and D also strongly induced mineralization of hBMMSC when it was co-treated with osteogenic induction cocktails (OIC) (FIG. 5). When various concentrations of Compound C and D treated, both compounds showed osteogenesis inducing activity (stimulation of mineralization) even at the 0.5 μM (FIG. 6). ALP mRNA expression was increased by treatment with Compound C and D in hBMMSC (FIG. 7).

### Example 3

### Modulating Activity of Cardiomyogenesis in Mouse Embryonic Stem Cells

The following compounds (Compound B and E) were used in this example

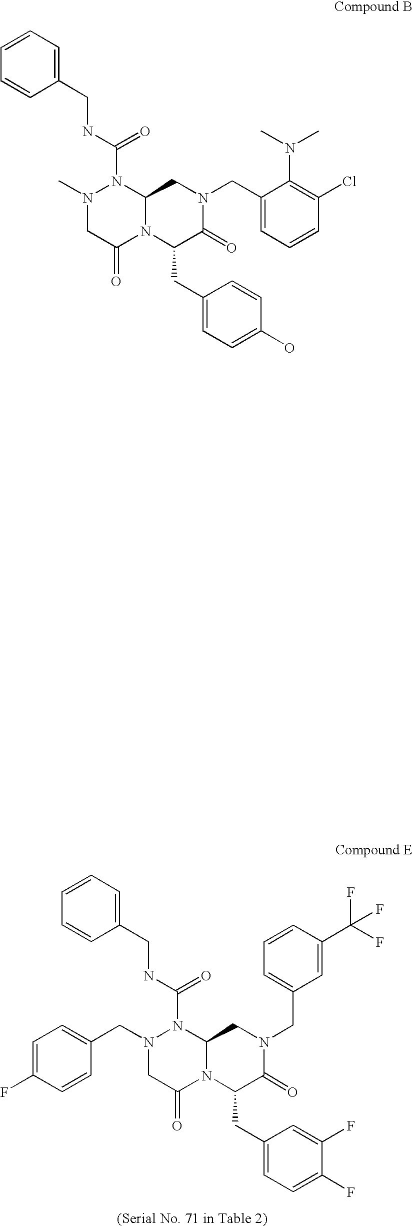

Methods

a. Cell culture: Mouse embryonic stem (ES) cells, TC1 derived from 129/S6 strain were cultured on irradiated feeder layer in the culture medium which is high-glucose DMEM (Gibco) BRL, Germany) supplemented with 15% FBS (Hyclone), 0.1 mM beta-mercaptoethanol, 1 μM sodium pyruvate, 1 mM L-glutamine and 1× non-essential amino acids.

b. Establishment of alpha-MHC stably transformed ES cell line: A construct (FIG. 8) containing 1.8 kb promoter sequence of the mouse alpha-MHC gene between EcoRI and SalI sites of pEGFP-1 (Clontech Laboratories, Inc, CA, USA) was linearized at XhoI site, and was introduced into mouse ES cells, TC1, using the Gene Pulser II (BioRad, MA). A selection medium containing G418 was applied for two weeks from 24 hours after electroporation. The selected ES cell clones were harvested and expanded on the feeder cell layer. The green fluorescence-expressing clones when differentiated into cardiomyocytes were used in this experiment.

c. Cardiomyogenesis modulating activity assay: Embryoid body (EB) was formed by hanging drop culture using about a number of 600 (six hundred) ES cells for two days and it was further expanded by culturing in suspension condition on a Petri-dish for another two days. Then each EB was plated on a well of 96 well culture plates. Test compounds were dissolved in DMSO and diluted with culture media (final 0.05% DMSO) and EB was treated with test compound for 5 days. Culture medium was changed every three days after plating. Expression level of EGFP was measured with FACS (Beckman). Fold change of EGFP expression level compared with DMSO control by each compound was summarized in Table 2 (Cardiomyogenesis). For FACS analysis, differentiating cells were dissociated with trypsin. For the determination of the extent of differentiation, EB was observed and photographed with an inverted fluorescence microscope (Zeiss, Germany). Number of beating EBs was counted under the microscope and recorded once daily.

d. Real time RT-PCR: To analyze the mRNA levels of cardiomyogenesis marker genes, Atrial Natriuretic Peptide (ANP) and Nk×2.5, total RNA was isolated using Trizol (Invitrogen-GIBCO-BRL, Baltimore, Md., USA) from stem cells with or without Compound E treatment for 7 days and 10 days. Relative expression level of each mRNA was measured.

Results

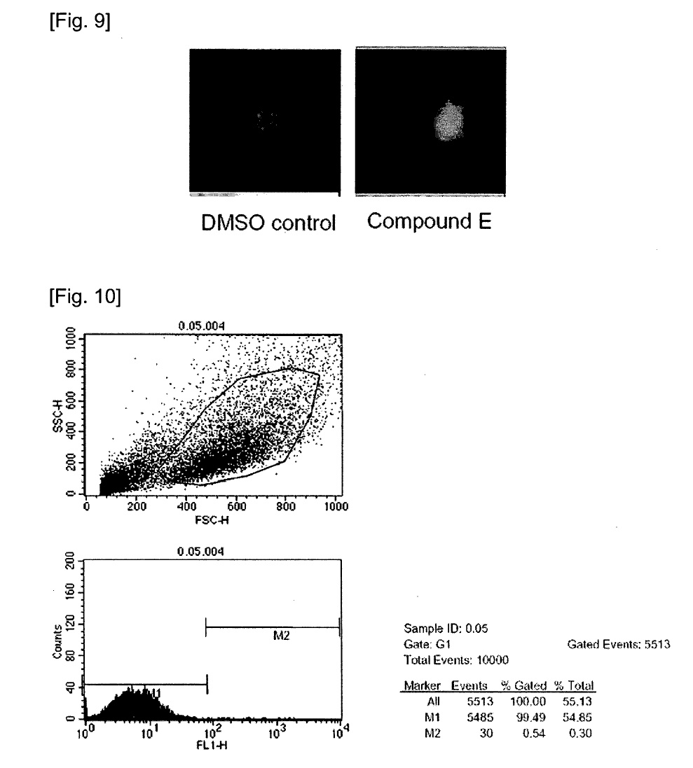

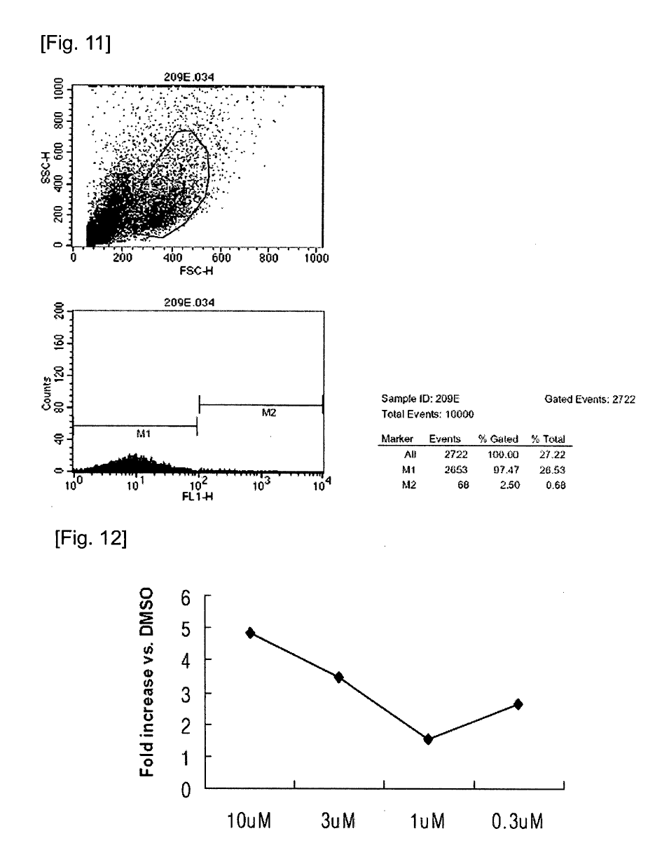

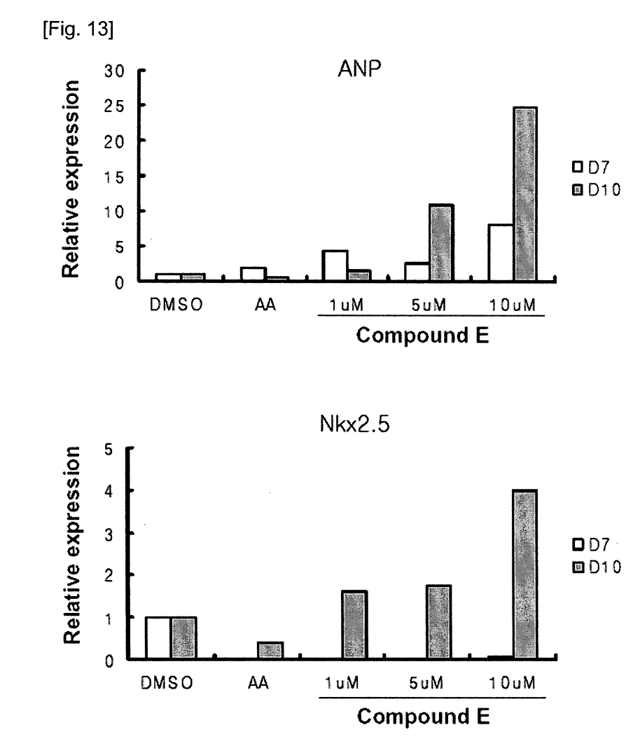

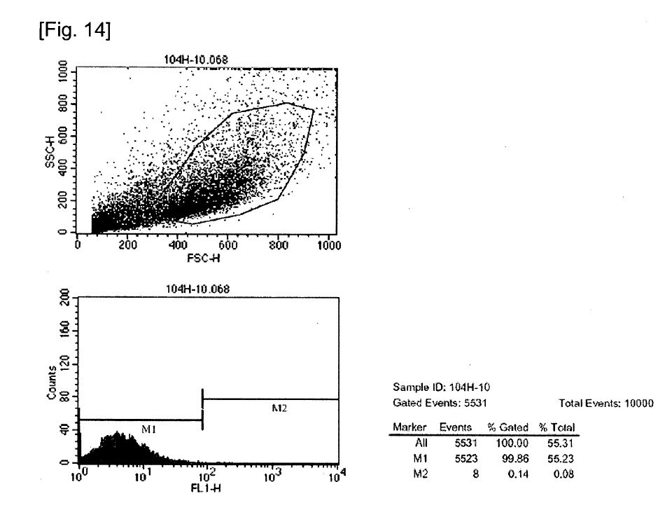

DMSO treated-control EB showed gradual increase of expression of fluorescence as time goes, but its expression level was minimal even at 5 days after incubation. Compound E treatment (10 μM) enhanced expression of fluorescence after treatment when it was observed under the fluorescence microscope. And its level was significantly enhanced at Day 5 compared with DMSO control (FIG. 9). FACS analysis of cell population of fluorescence expressing cells showed percentage of fluorescence positive (alpha-MHC positive) cell was increased 4.6 fold by Compound E treatment at 10 μM (FIG. 11) compared with the DMSO control (FIG. 10). And its cardiomyogenesis differentiation promoting activity was dose-dependent (FIG. 12). Compound E strongly increased the expression level of ANP and Nk×2.5 mRNA in the ES cells when it was treated for 7 and 10 days compared with DMSO control (FIG. 13). Compound B treatment reduced expression of fluorescence (0.26: 1=Compound B: DMSO control) when it was exposed to the mouse embryonic stem cells at 10 μM for 5 days (FIG. 14).

### Example 4

### Modulation of Myogenesis in C2C12 Myoblast Cells

The following compounds (Compounds B and F) were used in this example:

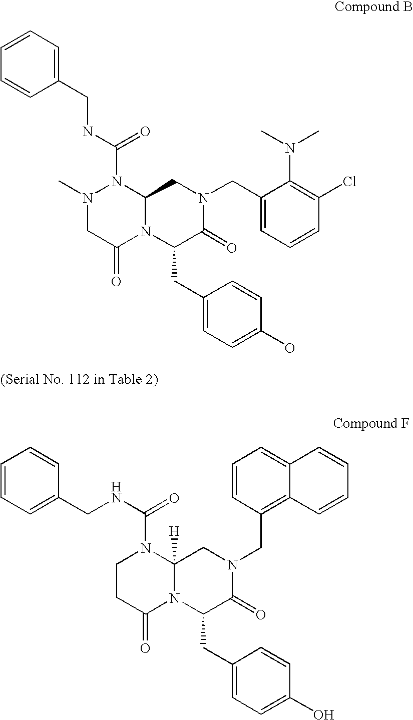

Materials and Methods

a. Cell culture: Murine C2C12 myoblastic cell line (satellite cells from thigh muscle) purchased from American Tissue Type Collection (ATCC) was maintained in exponential phase of growth using 10% FBS/DMEM with Glutamax designed as GM (growth medium) supplied with antibiotic solution (Penicillin and Streptomycin) in a controlled humidified air atmosphere supplied with 5% CO₂, at 37° C. in a multiwell or tissue culture Petri dishes (Corning-Costar Inc U.S.A.).

b. Induction of differentiation and test compound treatment: Every other day the cells were washed twice with phosphate buffered saline (PBS) and medium was changed until they reached 100% confluence. Confluent cells (myoblasts of the same cell density fully covering surface dish) were then guided to post mitotic status, and differentiation and fusion were initiated by replacing GM with 2% (v/v) horse serum HS/DMEM designed as DM (differentiating medium). In the above mentioned conditions C2C12 myoblasts easily and fully differentiate into myotubes, therefore we could follow up modifications of differentiation process during 5 subsequent days. During the study freshly prepared media without or with the experimental factors were changed every 24 hours. Test compounds were dissolved in DMSO and diluted with culture medium. Test compounds (if not specified, 10 μM was used, final DMSO 0.1% v/v) or DMSO (0.1% v/v) were added to the medium.

c. Expression of MyoD and Myf5: Cells were lysed and protein expression levels were analyzed by Western-immunoblot analysis, as described in the literature (Verma et al., British J Pharmacol, 2004, 143, 106-13). Equal amount of protein samples were resolved by sodium dodecyl sulphate poly-acrylamide gel electrophoresis (SDS-PAGE) and transferred to nitrocellulose membranes. The membranes were blocked with 5% bovine serum albumin (BSA) and incubated with the indicated primary antibodies for 12˜h: polyclonal C20 antion MyoD (from Santa-Cruz, Biotechnology, Santa Cruz, Calif.) diluted 1/400, polyclonal anti-Myf-5 directed against the COOH-terminal portion of the protein. After several washes in PBS, membranes were incubated with chemiluminescence reagents.

Results

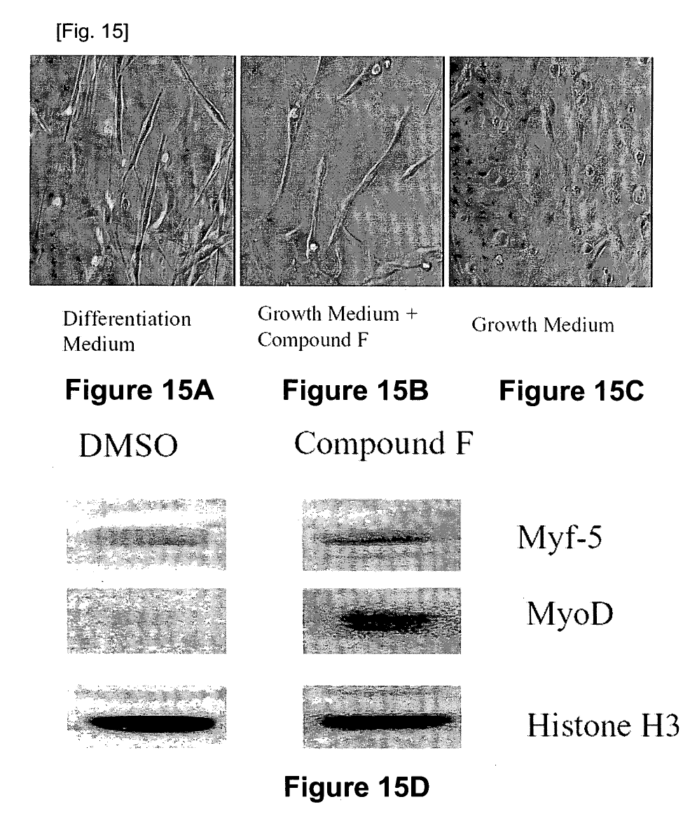

C2C12 is well-characterized cell culture model used to study skeletal muscle differentiation. Under conditions permissive for differentiation, such as low serum concentration, C2C12 myoblasts undergo differentiation to form myotubes. When these cells were incubated for 3 days in differentiation medium (DM) containing 2% horse serum, extensive myotube formation was observed in C2C12 cells. These myotube forming C2C12 cells showed a spindle shaped morphology and membrane fusion to form multinucleated myotubes (FIG. 15A) compared with cells grown in growth medium (GM) (FIG. 15C). When Compound F was added to the cells grown in GM, it caused extensive myotube formation (FIG. 15B). Also MyoD and Myf5 protein expression level was significantly increased by the treatment of Compound F as shown in FIG. 15D.

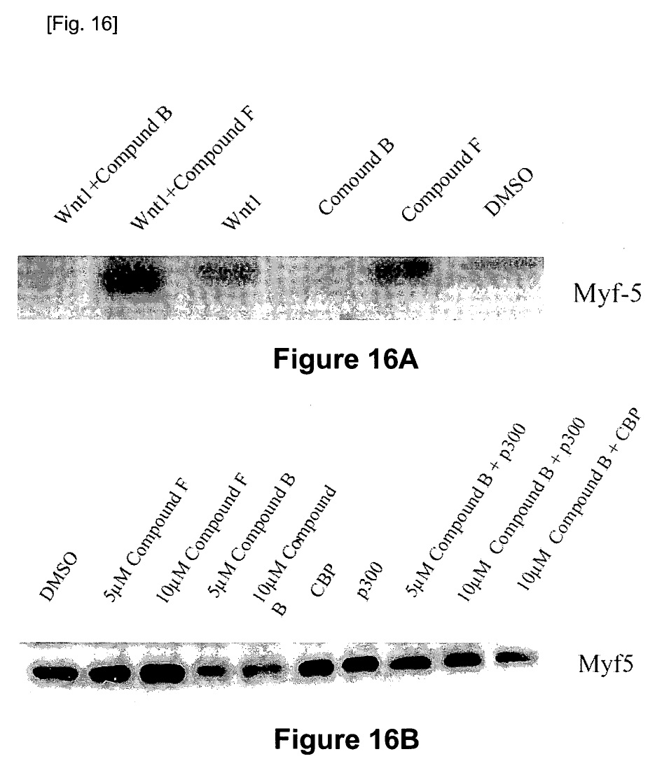

Wnt/beta-catenin signaling plays important roles in myogenic fate determination and differentiation (Pan et al., PNAS 2005, 102, 17378-83). To investigate the relation between Wnt pathway modulating compounds and Wnt ligand in myogenesis, Wnt1 conditioned medium was treated with or without test Compound B and F. Myf-5 expression was increased by the treatment of Wnt1 and its expression level was further enhanced by co-treatment of Compound F. Compound B treatment reduced expression of Myf-5 in C2C12 cells and it also abolished Myf-5 enhancing activity of Wnt1 when it was co-treated with Wnt1 (FIG. 16A).

CREB-binding protein (CBP) and or its closely related homolog, p300 is believed to participate in the activities of a lot of different transcription factors including TCF4/beta-catenin signaling. To evaluate the possible interaction between CBP or p300 with Wnt pathway modulating test compounds, CBP or p300 with or without test compounds were exposed to the C2C12 cells and cellular expression level of Myf5 protein was determined. Compound F at 5 and 10 μM dose-dependently increased expression of Myf5 compared with DMSO control. Compound B at 5 μM and 10M dose-dependently decreased expression of Myf5 compared with DMSO control. And co-treatment of p300 with Compound B p300 rescues the decrease of Myf5 expression by Compound B but it was not changed by co-treatment of CBP (FIG. 16B). This suggests that Compound B blocks or competes with p300 in the same signaling pathway but not with closely related analog, CBP.

1. A composition comprising a compound having the following general formula (I) for inducing or inhibiting differentiation of stem cells: 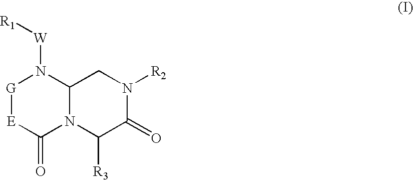
wherein:
E is -(ZR₄)— or —(C═O)—;
G is nothing, —(XR₅)—, or —(C═O)—;
W is —Y(C═O)—, —(C═O)NH—, —(SO₂)— or nothing;
Y is oxygen or sulfur;
X or Z is independently nitrogen or CH; and
R₁, R₂, R₃, R₄, R₅ are the same or different and independently selected from the group consisting of:
an amino acid side chain moiety;
C₁₋₁₂ alkyl or substituted C₁₋₁₂ alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;
C₁₋₆alkoxy;
C₆₋₁₂aryl or substituted C₆₋₁₂aryl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl;
monocyclic aryl-alkyl having 5 to 7 ring members, which may have 1 to 2 heteroatoms selected from nitrogen, oxygen or sulfur, or substituted monocyclic aryl-alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
bicyclic aryl-alkyl having 8 to 10 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;
tricyclic aryl-alkyl having 5 to 14 ring members, which may have 1 to 2 heteroatoms selected from nitrogen, oxygen or sulfur, or substituted bicyclic aryl-alkyl having one or more substituents independently selected from halogen, C₁₋₆alkyl, C₁₋₆alkoxy, cyano, hydroxyl;
arylC₁₋₄alkyl or substituted arylC₁₋₄alkyl having one or more substituents independently selected from amino, amidino, guanidino, hydrazino, C₁₋₄alkylamino, C₁₋₄dialkylamino, C₃₋₆cycloalkyl, halogen, perfluoroC₁₋₄alkyl, C₁₋₆alkyl, C₁₋₃alkoxy, nitro, carboxyl, cyano, sulfuryl, hydroxyl, amide, C₁₋₆alkyloxyC₁₋₆acyl and morphorlinylC₁₋₆alkyl;
cycloalkylalkyl or substituted cycloalkylalkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl; and
cycloalkyl or substituted cycloalkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl.

2. The composition comprising the compound according to claim 1, wherein R₁, R₂, R₃, R₄, R₅ are the same or different and independently selected from the group consisting of:
C₁₋₁₂ alkyl or substituted C₁₋₁₂ alkyl having one or more substituents independently selected from amino, guanidino, C₁₋₄alkylguanidino, diC₁₋₄alkylguanidino, amidino, C₁₋₄alkylamidino, diC₁₋₄alkylamidino, C₁₋₅alkylamino, diC₁₋₅alkylamino, sulfide, carboxyl, hydroxyl;
C₁₋₆alkoxy;
cycloalkylC₁₋₃alkyl;
cycloalkyl;
phenyl or substituted phenyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
phenylC₂₋₄alkyl or phenylC₂₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, sulfide, hydroxyl;
naphthyl or substituted naphthyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
naphthylC₁₋₄alkyl or naphthylC₁₋₄alkyl having one or more substituents independently selected from amino, amidino, guanidino, hydrazino, C₁₋₄alkylamino, C₁₋₄dialkylamino, halogen, perfluoroC₁₋₄alkyl, C₁₋₆alkyl, C₁₋₃alkoxy, nitro, carboxyl, cyano, sulfuryl, hydroxyl;
benzyl or substituted benzyl having one or more substituents independently selected from amino, amidino, guanidino, hydrazino, C₁₋₄alkylamino, C₁₋₄dialkylamino, halogen, perfluoro C₁₋₄alkyl, trifluoroC₁₋₄alkyl, C₁₋₆alkyl, C₁₋₃alkoxy, nitro, carboxyl, cyano, sulfuryl and hydroxyl;
bisphenylmethyl or substituted bisphenylmethyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
benzylphenyl amide, or substituted benzylphenyl amide having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
pyridyl or substituted pyridyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
pyridylC₁₋₄alkyl, or substituted pyridylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₄alkyl, C₁₋₃alkoxy, nitro, carboxyl, cyano, sulfuryl and hydroxyl;
pyrimidylC₁₋₄alkyl, or substituted pyrimidylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxyl, cyano, sulfuryl and hydroxyl;
triazin-2-ylC₁₋₄alkyl, or substituted triazin-2-ylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
imidazolylC₁₋₄alkyl or substituted imidazolylC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
benzothiazolinC₁₋₄alkyl or substituted benzothiazolinC₁₋₄alkyl having one or more substituents independently selected from amino, amidino, guanidino, hydrazino, C₁₋₄alkylamino, C₁₋₄dialkylamino, halogen, perfluoro C₁₋₄alkyl, C₁₋₆alkyl, C₁₋₃alkoxy, nitro, carboxy, cyano, sulfuryl and hydroxyl;
phenoxazinC₁₋₄alkyl;
benzyl p-tolyl ether;
phenoxybenzyl;
N-amidinopiperazinyl-N—C₁₋₄alkyl;
quinolineC₁₋₄alkyl;
N-amidinopiperazinyl;
N-amidinopiperidinylC₁₋₄alkyl;
4-aminocyclohexylC₁₋₂alkyl; and
4-aminocyclohexyl.

3. The composition comprising the compound according to claim 1, wherein E is -(ZR₄)- and G is —(XR₅)—, wherein Z is CH and X is nitrogen, and the compound has a structure of Formula (II):

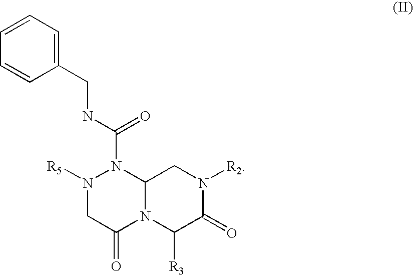

4. The composition comprising the compound according to claim 3, wherein the compound has a structure of Formula (III):

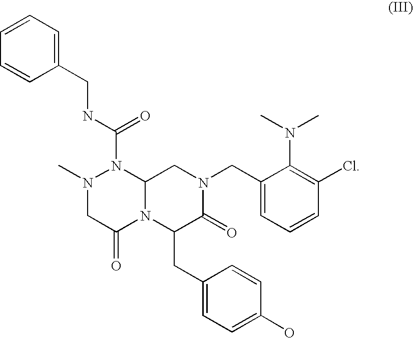

5. The composition comprising the compound according to claim 1, wherein E is -(ZR₄)- and G is nothing, wherein Z is nitrogen, and the compound has a structure of Formula (IV):

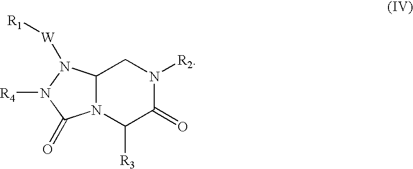

6. The composition comprising the compound according to claim 1, wherein E is -(ZR₄)— and G is —(XR₅)—, wherein Z and X are independently CH, and the compound has a structure of Formula (V):

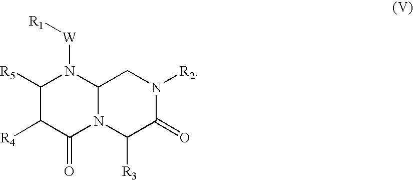

7. The composition comprising the compound according to claim 6, wherein the compound has a structure of Formula (VI):

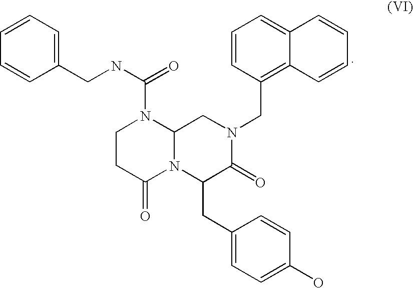

8. A method for inducing or inhibiting differentiation of a stem cell, comprising contacting the stem cells with an amount of a composition according to claim 1, wherein the amount is effective to modulate differentiation of the stem cells.

9. The method according to claim 8, for inducing or inhibiting one selected from the group consisting of osteogenesis, cardiomyogenesis, myogenesis.

10. The method according to claim 8, for inducing or inhibiting one selected from the group consisting of neurogenesis and hematopoiesis.

11. A method for inhibiting proliferation or self-renewal of cancer stem cell or cancer initiating cell, comprising contacting the stem cell with an amount of a compound according to claim 1, wherein the amount is effective to inhibit proliferation or self-renewal of cancer stem cell or cancer initiating cell.

12. The method according to claim 8, wherein said stem cell is cultured in a media with 1 nM˜50 μM of said compound.

13. A pharmaceutical composition comprising a compound according to claim 1 and pharmaceutically acceptable carrier.

14. The pharmaceutical composition according to claim 13, for treatment of medical conditions selected from the group consisting of osteoporosis, bone fracture, bone injuries, myocardiac infarction, cardiomyopathy, degenerative muscle diseases, and urinary incontinence.

15. The pharmaceutical composition according to claim 13, for selective killing, inhibition or modulation of the growth of cancer stem cell present within a cancerous tumor by administering said pharmaceutical composition to a subject.

16. The composition according to claim 6 for induction of myogenesis.
